(19) United States
(12) Patent Application Publication
(10) Pub. No.: US 2017/0173070 A1
(43) Pub. Date: Jun. 22, 2017

(54) METHODS OF ADMINISTERING LMWH

(71) Applicant: MOMENTA PHARMACEUTICALS, INC., Cambridge, MA (US)

(72) Inventors: Martijn Lolkema, Utrecht (NL); Birgit Schultes, Arlington, MA (US); Joel Pradines, Watertown, MA (US)

(21) Appl. No.: 15/300,425

(22) PCT Filed: Apr. 3, 2015
(86) PCT No.: PCT/US15/24296

§ 371 Date: Sep. 29, 2016

Related U.S. Application Data
(60) Provisional application No. 61/975,556, filed on Apr. 4, 2014.

(51) Int. Cl. A61K 31/727; A61K 31/7068; A61K 45/06

(52) U.S. Cl. CPC A61K 31/727; A61K 45/06; A61K 31/7068

(57) ABSTRACT

Methods of treating cancer comprising administering a LMWH.

### RELATED APPLICATIONS

This application claims the benefit of U.S. Provisional Application No. 61/975,556, filed Apr. 4, 2014, the contents of which are hereby incorporated by reference in their entirety.

### BACKGROUND

This disclosure relates to methods of administering LMWHs, e.g., M402.

### SUMMARY OF THE INVENTION

In one aspect, disclosed herein are, inter alia, methods of treating a cancer in a subject, the method comprising administering at least one dose of a LMWH preparation (e.g., a M402 preparation) described herein, and administering an initial dose of an anti-cancer treatment (e.g., a chemotherapeutic agent) that increases wnt/β-catenin signaling, wherein the LMWH preparation (e.g., the M402 preparation) is administered prior to the administration of the initial dose of the anti-cancer treatment (e.g., the chemotherapeutic agent).

In some embodiments, the LMWH preparation (e.g., the M402 preparation) is administered at least 1 hour (e.g., at least 2 hours, 3 hours, 4 hours, 5 hours, 6 hours, 12 hours, 24 hours, 37 hours, 48 hours) prior to the administration of the initial dose of the anti-cancer treatment (e.g., the chemotherapeutic agent). In some embodiments, the LMWH preparation (e.g., the M402 preparation), is administered at least 1 day (e.g., at least 2 days, 3 days, 4 days, 5 days, 6 days, 7 days) prior to the administration of the initial dose of the anti-cancer treatment (e.g., chemotherapeutic agent). In some embodiments, the LMWH preparation (e.g., the M402 preparation), is administered at least 1 week (e.g., at least 2 weeks, 3 weeks, 4 weeks, 5 weeks, 6 weeks) prior to the administration of the initial dose of the anti-cancer treatment (e.g., chemotherapeutic agent). In some embodiments, the LMWH preparation (e.g., the M402 preparation), is administered at least 1 month (e.g., at least 2 months, 3 months, 4 months, 5 months, 6 months) prior to the administration of the initial dose of the anti-cancer treatment (e.g., chemotherapeutic agent). In some embodiments, the LMWH preparation (e.g., the M402 preparation), is administered within 6 hours or less (e.g., 5 hours or less; 4 hours or less; 3 hours or less; 2 hours or less; 1 hour or less) of the administration of the initial dose of the anti-cancer treatment (e.g., chemotherapeutic agent).

In some embodiments, more than one dose (e.g., 2, 3, 4, 5, 6, 7, 8, 9, 10 or more doses) of the LMWH preparation (e.g., the M402 preparation), is administered to the subject.

In some embodiments, the LMWH preparation (e.g., the M402 preparation) is administered daily (e.g., once daily, twice daily), weekly, every other week, every three weeks, monthly, every other month, or every three months for a period of at least 1 week (e.g., at least 2 weeks, 3 weeks, 4 weeks, 5 weeks, 6 weeks, 7 weeks, 1 month, 2 months, 3 months, 4 months, 5 months, 6 months, 1 year). In some embodiments, the LMWH preparation (e.g., the M402 preparation), is administered daily, e.g., once daily. In some embodiments, the LMWH preparation (e.g., the M402 preparation), is administered daily, e.g., once daily, for a period of at least 1 week (e.g., at least 2 weeks, 3 weeks, 4 weeks, 5 weeks, 6 weeks, 7 weeks, 1 month, 2 months, 3 months, 4 months, 5 months, 6 months, 1 year). In some embodiments, the LMWH preparation (e.g., the M402 preparation), is administered weekly, e.g., once weekly. In some embodiments, the LMWH preparation (e.g., the M402 preparation), is administered weekly, e.g., once weekly, for a period of at least 1 week (e.g., at least 2 weeks, 3 weeks, 4 weeks, 5 weeks, 6 weeks, 7 weeks, 1 month, 2 months, 3 months, 4 months, 5 months, 6 months, 1 year). In some embodiments, the LMWH preparation (e.g., the M402 preparation), is administered according to a dosing regimen for M402.

In some embodiments, the method comprises administering the LMWH preparation (e.g., the M402 preparation) at one or more dose, at a predetermined interval between doses, wherein the initial dose is administered at least 1 hour (e.g., at least 2 hours, at least 3 hours, at least 6 hours, at least 12 hours, at least 24 hours, at least 48 hours, at least 73 hours; at least 1 week, at least 2 weeks, at least 3 weeks, at least 1 month, at least 2 months, at least 3 months, at least 4 months, at least 5 months, at least 6 months) prior to the administration of the anti-cancer treatment (e.g., the chemotherapeutic agent). In some embodiments, the predetermined interval between doses is at least 6 hours, 12 hours, 24 hours, or 48 hours. In some embodiments, the LMWH preparation (e.g., the M402 preparation) is administered at least 1, 2, 3, 4, 5, 6, or 7 times per week. In some embodiments, the LMWH preparation (e.g., the M402 preparation) is administered at least 3 or 4 times per week, wherein the predetermined interval between doses is at least 24 hours.

In some embodiments, the anti-cancer treatment (e.g., the chemotherapeutic agent) is administered daily (e.g., once daily, twice daily), weekly, every other week, every three weeks, monthly, every other month, or every three months.

In some embodiments, the anti-cancer treatment (e.g., the chemotherapeutic agent) is administered daily (e.g., once daily, twice daily), weekly, every other week, every three weeks, monthly, every other month, or every three months for a period of at least 1 week (e.g., at least 2 weeks, 3 weeks, 4 weeks, 5 weeks, 6 weeks, 7 weeks, 1 month, 2 months, 3 months, 4 months, 5 months, 6 months, 1 year).

In some embodiments, the anti-cancer treatment (e.g., the chemotherapeutic agent) is administered daily (e.g., once daily, twice daily), weekly, every other week, every three weeks, monthly, every other month, or every three months for a period of at least 1 week (e.g., at least 2 weeks, 3 weeks, 4 weeks, 5 weeks, 6 weeks, 7 weeks, 1 month, 2 months, 3 months, 4 months, 5 months, 6 months, 1 year), in combination with another chemotherapeutic agent that is administered daily (e.g., once daily, twice daily), weekly, every other week, every three weeks, monthly, every other month, or every three months for a period of at least 1 week (e.g., at least 2 weeks, 3 weeks, 4 weeks, 5 weeks, 6 weeks, 7 weeks, 1 month, 2 months, 3 months, 4 months, 5 months, 6 months, 1 year).

In some embodiments, the anti-cancer treatment (e.g., the chemotherapeutic agent, e.g., gemcitabine) is administered once a week for the first 7 weeks; followed by one week with no administration of the chemotherapeutic agent; followed by administration of the chemotherapeutic agent once a week for 3 weeks and no administration for one week of a 28 day cycle, e.g., wherein the chemotherapeutic agent is administered for at least 1, 3, 4, 5, 6, 7, 8, 9, 10, or 20 twenty eight day cycles.

In some embodiments, the anti-cancer treatment (e.g., the chemotherapeutic agent, e.g., gemcitabine) is administered once a week for 3 weeks and one week with no administration of a 28 day cycle, e.g., for a period of at least 4 weeks (e.g., at least 5 weeks, 6 weeks, 7 weeks, 1 month, 2 months, 3 months, 4 months, 5 months, 6 months, 1 year, 2 years, 3 years, 4 years, 5 years, 6 years, 7 years, 8 years, 9 years, 10 years, 1 cycle, 2 cycles, 3 cycles, 4 cycles, 5 cycles, 6 cycles, 7 cycles, 8 cycles, 10 cycles, 20 cycles).

In some embodiments, the anti-cancer treatment (e.g., the chemotherapeutic agent, e.g., gemcitabine) is administered on days 1 and 8 of a 21 day cycle, e.g., for a period of at least 3 weeks (e.g., at least 4 weeks, 5 weeks, 6 weeks, 7 weeks, 1 month, 2 months, 3 months, 4 months, 5 months, 6 months, 1 year, 2 years, 3 years, 4 years, 5 years, 6 years, 7 years, 8 years, 9 years, 10 years, 1 cycle, 2 cycles, 3 cycles, 4 cycles, 5 cycles, 6 cycles, 7 cycles, 8 cycles, 10 cycles, 20 cycles).

In some embodiments, the anti-cancer treatment (e.g., the chemotherapeutic agent, e.g., gemcitabine) is administered on days 1, 8, and 15 of each of a 28 day cycle, e.g., for a period of at least 4 weeks (e.g., at least 5 weeks, 6 weeks, 7 weeks, 1 month, 2 months, 3 months, 4 months, 5 months, 6 months, 1 year, 2 years, 3 years, 4 years, 5 years, 6 years, 7 years, 8 years, 9 years, 10 years, 1 cycle, 2 cycles, 3 cycles, 4 cycles, 5 cycles, 6 cycles, 7 cycles, 8 cycles, 10 cycles, 20 cycles).

In some embodiments, the anti-cancer agent (e.g., the chemotherapeutic agent, e.g., gemcitabine) is administered at a dosing regimen for gemcitabine.

In some embodiments, the LMWH preparation (e.g., the M402 preparation), is administered subcutaneously.

In some embodiments, the LMWH preparation (e.g., the M402 preparation), is administered at a dose from 1 to 40 mg/kg (e.g., 1-20 mg/kg, 1-10 mg/kg, 1-5 mg/kg, 1-3 mg/kg). In some embodiments, the LMWH preparation (e.g., the M402 preparation), is administered at a dose of 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 20 mg/kg or 40 mg/kg. In some embodiments, the LMWH preparation (e.g., the M402 preparation), is administered at a dose of 1 mg/kg. In some embodiments, the LMWH preparation (e.g., the M402 preparation), is administered at a dose of 2 mg/kg. In some embodiments, the LMWH preparation (e.g., the M402 preparation), is administered at a dose of 4 mg/kg. In some embodiments, the LMWH preparation (e.g., the M402 preparation), is administered at a dose of 6 mg/kg. In some embodiments, the LMWH preparation (e.g., the M402 preparation), is administered at a dose of 8 mg/kg.

In some embodiments, the anti-cancer treatment (e.g., the chemotherapeutic agent) is administered intravenously, orally, or subcutaneously. In some embodiments, the anti-cancer treatment (e.g., the chemotherapeutic agent) is administered intravenously.

In some embodiments, the anti-cancer treatment (e.g., the chemotherapeutic agent) is administered in combination with a second anti-cancer treatment (e.g., a second chemotherapeutic agent). In some embodiments, the anti-cancer treatment (e.g., the chemotherapeutic agent) is gemcitabine, and the second anti-cancer agent (e.g., the second chemotherapeutic agent) is abraxane.

In some embodiments, gemcitabine is administered at a dosing regimen for gemcitabine and abraxane is administered at a dosing regimen for abraxane.

In some embodiments, the cancer is selected from the group consisting of pancreatic cancer, breast cancer, triple negative breast cancer, inflammatory breast cancer, esophageal cancer, glioma, stomach cancer, lung cancer, and colorectal cancer. In some embodiments, the cancer is pancreatic cancer. In some embodiments, the cancer is a metastatic cancer. In some embodiments, the cancer is metastatic pancreatic cancer. In some embodiments, the cancer is one in which the standard of care for the cancer is associated with the inducement of one or more of the following properties in at least one cancer cell: (a) a mesenchymal cell phenotype (e.g., as characterized by (e.g., increased vimentin expression and/or decreased E-cadherin expression compared to a reference standard (e.g., expression of the marker in a sample from a cancer which has not been treated with the standard of care))); (b) a cancer stem cell phenotype (e.g., as characterized by (e.g., increased expression of one or more embryonically expressed gene (e.g., increased SOX2, OCT-4, and/or Nanog expression compared to a reference standard (e.g., expression of the marker in a sample from a cancer which has not been treated with the standard of care)), increased CD133, CD44, CD24, THY1, EpCAM, ALDH, and/or ABCB5 expression; compared to a reference standard (e.g., expression of the marker in a sample from a cancer which has not been treated with the standard of care))); or (c) an invasive or metastatic cancer cell phenotype (e.g., as characterized by (e.g., increased AKT signaling, increased Rac signaling, increased Rho signaling, increased ROBO signaling, increased expression of one or more gene associated with metastasis; compared to a reference standard (e.g., expression of the marker in a sample from a cancer which has not been treated with the standard of care))). In some embodiments, the standard of care is an antimetabolite (e.g., gemcitabine) at a dosing regimen for that antimetabolite (e.g., gemcitabine). In some embodiments, the standard of care is a platinum based agent (e.g., carboplatin, cisplatin) at a dosing regimen for that platinum based agent (e.g., carboplatin, cisplatin). In some embodiments, the standard of care is a taxane (e.g., docetaxel) at a dosing regimen for that taxane (e.g., docetaxel).

In some embodiments, administration of the anti-cancer treatment (e.g., the chemotherapeutic agent) is associated with the cancer becoming refractory, relapsed, and/or resistant to the anti-cancer treatment (e.g., the chemotherapeutic agent).

Also disclosed herein are methods of treating a cancer in a subject, the method comprising administering a LMWH preparation (e.g., a M402 preparation) described herein, and administering an initial dose of an anti-cancer treatment (e.g., a chemotherapeutic agent), wherein the LMWH preparation (e.g., the M402 preparation) is administered once a week for two weeks; optionally followed by 1 week with no administration of the LMWH nor the anti-cancer treatment; followed by co-administration of the LMWH and the chemotherapeutic agent, wherein the LMWH preparation is administered once a week, for a period of at least 12 weeks (e.g., at least 3 months, 4 months, 5 months, 6 months, 1 year, 2 years, 3 years, 4 years, 5 years, 6 years, 7 years, 8 years, 9 years, 10 years, 1 cycle, 2 cycles, 3 cycles, 4 cycles, 5 cycles, 6 cycles, 7 cycles, 8 cycles, 10 cycles, 20 cycles); and wherein the chemotherapeutic agent administered once a week for the first 7 weeks; followed by one week with no administration of the chemotherapeutic agent; followed by administration of the chemotherapeutic agent once a week for 3 weeks and no administration for one week of a 28 day cycle, e.g., wherein the chemotherapeutic agent is administered for at least 1, 3, 4, 5, 6, 7, 8, 9, 10, or 20 twenty eight day cycles. In some embodiments, the anti-cancer treatment (e.g., the chemotherapeutic agent) increases wnt/β-catenin signaling.

Also disclosed herein are methods of treating a cancer in a subject, the method comprising administering a LMWH preparation (e.g., a M402 preparation) described herein, and administering an initial dose of a chemotherapeutic agent, wherein the LMWH preparation (e.g., the M402 preparation) is administered prior to the administration of the initial dose of the chemotherapeutic agent; wherein the chemotherapeutic agent is administered once a week for the first 7 weeks; followed by one week with no administration of the chemotherapeutic agent; followed by administration of the chemotherapeutic agent once a week for 3 weeks and no administration for one week of a 28 day cycle, e.g., wherein the chemotherapeutic agent is administered for at least 1, 3, 4, 5, 6, 7, 8, 9, 10, or 20 twenty eight day cycles. In some embodiments, the anti-cancer treatment (e.g., the chemotherapeutic agent) increases wnt/β-catenin signaling. In some embodiments, the cancer is pancreatic cancer (e.g., metastatic pancreatic cancer).

Also disclosed herein are methods of treating a cancer in a subject, the method comprising administering at least one dose of a LMWH preparation (e.g., a M402 preparation) described herein, and administering an initial dose of an anti-cancer treatment, wherein the LMWH preparation (e.g., the M402 preparation) is administered within 1 hour before or after the administration of the initial dose of the anti-cancer treatment (e.g., the chemotherapeutic agent). In some embodiments, the anti-cancer treatment (e.g., the chemotherapeutic agent) increases wnt/β-catenin signaling.

In some embodiments, more than one dose (e.g., 2, 3, 4, 5, 6, 7, 8, 9, 10 or more doses) of the LMWH preparation (e.g., the M402 preparation), is administered to the subject.

In some embodiments, the LMWH preparation (e.g., the M402 preparation) is administered daily (e.g., once daily, twice daily), weekly, every other week, every three weeks, monthly, every other month, or every three months for a period of at least 1 week (e.g., at least 2 weeks, 3 weeks, 4 weeks, 5 weeks, 6 weeks, 7 weeks, 1 month, 2 months, 3 months, 4 months, 5 months, 6 months, 1 year). In some embodiments, the LMWH preparation (e.g., the M402 preparation), is administered daily, e.g., once daily. In some embodiments, the LMWH preparation (e.g., the M402 preparation), is administered daily, e.g., once daily, for a period of at least 1 week (e.g., at least 2 weeks, 3 weeks, 4 weeks, 5 weeks, 6 weeks, 7 weeks, 1 month, 2 months, 3 months, 4 months, 5 months, 6 months, 1 year). In some embodiments, the LMWH preparation (e.g., the M402 preparation), is administered weekly, e.g., once weekly. In some embodiments, the LMWH preparation (e.g., the M402 preparation), is administered weekly, e.g., once weekly, for a period of at least 1 week (e.g., at least 2 weeks, 3 weeks, 4 weeks, 5 weeks, 6 weeks, 7 weeks, 1 month, 2 months, 3 months, 4 months, 5 months, 6 months, 1 year).

In some embodiments, the LMWH preparation (e.g., the M402 preparation), is administered according to a dosing regimen for M402.

In some embodiments, the methods further comprise administering the LMWH preparation (e.g., the M402 preparation) at one or more dose, at a predetermined interval between doses. In some embodiments, the predetermined interval between doses is at least 6 hours, 12 hours, 24 hours, or 48 hours. In some embodiments, the LMWH preparation (e.g., the M402 preparation) is administered at least 1, 2, 3, 4, 5, 6, or 7 times per week. In some embodiments, the LMWH preparation (e.g., the M402 preparation) is administered at least 3 or 4 times per week, wherein the predetermined interval between doses is at least 24 hours.

In some embodiments, the anti-cancer treatment (e.g., the chemotherapeutic agent) is administered daily (e.g., once daily, twice daily), weekly, every other week, every three weeks, monthly, every other month, or every three months. In some embodiments, the anti-cancer treatment (e.g., the chemotherapeutic agent) is administered daily (e.g., once daily, twice daily), weekly, every other week, every three weeks, monthly, every other month, or every three months for a period of at least 1 week (e.g., at least 2 weeks, 3 weeks, 4 weeks, 5 weeks, 6 weeks, 7 weeks, 1 month, 2 months, 3 months, 4 months, 5 months, 6 months, 1 year).

In some embodiments, the anti-cancer treatment (e.g., the chemotherapeutic agent) is administered daily (e.g., once daily, twice daily), weekly, every other week, every three weeks, monthly, every other month, or every three months for a period of at least 1 week (e.g., at least 2 weeks, 3 weeks, 4 weeks, 5 weeks, 6 weeks, 7 weeks, 1 month, 2 months, 3 months, 4 months, 5 months, 6 months, 1 year), in combination with another chemotherapeutic agent that is administered daily (e.g., once daily, twice daily), weekly, every other week, every three weeks, monthly, every other month, or every three months for a period of at least 1 week (e.g., at least 2 weeks, 3 weeks, 4 weeks, 5 weeks, 6 weeks, 7 weeks, 1 month, 2 months, 3 months, 4 months, 5 months, 6 months, 1 year).

In some embodiments, the anti-cancer treatment (e.g., the chemotherapeutic agent, e.g., gemcitabine) is administered once a week for the first 7 weeks; followed by one week with no administration of the chemotherapeutic agent; followed by administration of the chemotherapeutic agent once a week for 3 weeks and no administration for one week of a 28 day cycle, e.g., wherein the chemotherapeutic agent is administered for at least 1, 3, 4, 5, 6, 7, 8, 9, 10, or 20 twenty eight day cycles.

In some embodiments, the anti-cancer treatment (e.g., the chemotherapeutic agent, e.g., gemcitabine) is administered once a week for 3 weeks and one week with no administration of a 28 day cycle, e.g., for a period of at least 4 weeks (e.g., at least 5 weeks, 6 weeks, 7 weeks, 1 month, 2 months, 3 months, 4 months, 5 months, 6 months, 1 year, 2 years, 3 years, 4 years, 5 years, 6 years, 7 years, 8 years, 9 years, 10 years, 1 cycle, 2 cycles, 3 cycles, 4 cycles, 5 cycles, 6 cycles, 7 cycles, 8 cycles, 10 cycles, 20 cycles).

In some embodiments, the anti-cancer treatment (e.g., the chemotherapeutic agent, e.g., gemcitabine) is administered on days 1 and 8 of a 21 day cycle, e.g., for a period of at least 3 weeks (e.g., at least 4 weeks, 5 weeks, 6 weeks, 7 weeks, 1 month, 2 months, 3 months, 4 months, 5 months, 6 months, 1 year, 2 years, 3 years, 4 years, 5 years, 6 years, 7 years, 8 years, 9 years, 10 years, 1 cycle, 2 cycles, 3 cycles, 4 cycles, 5 cycles, 6 cycles, 7 cycles, 8 cycles, 10 cycles, 20 cycles).

In some embodiments, the anti-cancer treatment (e.g., the chemotherapeutic agent, e.g., gemcitabine) is administered on days 1, 8, and 15 of each of a 28 day cycle, e.g., for a period of at least 4 weeks (e.g., at least 5 weeks, 6 weeks, 7 weeks, 1 month, 2 months, 3 months, 4 months, 5 months, 6 months, 1 year, 2 years, 3 years, 4 years, 5 years, 6 years, 7 years, 8 years, 9 years, 10 years, 1 cycle, 2 cycles, 3 cycles, 4 cycles, 5 cycles, 6 cycles, 7 cycles, 8 cycles, 10 cycles, 20 cycles).

In some embodiments, the anti cancer agent (e.g., the chemotherapeutic agent, e.g., gemcitabine) is administered at a dosing regimen for gemcitabine.

In some embodiments, the LMWH preparation (e.g., the M402 preparation), is administered subcutaneously.

In some embodiments, the LMWH preparation (e.g., the M402 preparation), is administered at a dose from 1 to 40 mg/kg (e.g., 1-20 mg/kg, 1-10 mg/kg, 1-5 mg/kg, 1-3 mg/kg). In some embodiments, the LMWH preparation (e.g., the M402 preparation), is administered at a dose of 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 20 mg/kg or 40 mg/kg. In some embodiments, the LMWH preparation (e.g., the M402 preparation), is administered at a dose of 1 mg/kg. In some embodiments, the LMWH preparation (e.g., the M402 preparation), is administered at a dose of 2 mg/kg. In some embodiments, the LMWH preparation (e.g., the M402 preparation), is administered at a dose of 4 mg/kg. In some embodiments, the LMWH preparation (e.g., the M402 preparation), is administered at a dose of 6 mg/kg. In some embodiments, the LMWH preparation (e.g., the M402 preparation), is administered at a dose of 8 mg/kg.

In some embodiments, the anti-cancer treatment (e.g., the chemotherapeutic agent) is administered intravenously, orally, or subcutaneously. In some embodiments, the anti-cancer treatment (e.g., the chemotherapeutic agent) is administered intravenously.

In some embodiments, the anti-cancer treatment (e.g., the chemotherapeutic agent) is administered in combination with a second anti-cancer treatment (e.g., a second chemotherapeutic agent). In some embodiments, the anti-cancer treatment (e.g., the chemotherapeutic agent) is gemcitabine, and the second anti-cancer agent (e.g., the second chemotherapeutic agent) is abraxane.

In some embodiments, gemcitabine is administered at a dosing regimen for gemcitabine and abraxane is administered at a dosing regimen for abraxane.

In some embodiments, the cancer is selected from the group consisting of pancreatic cancer, breast cancer, triple negative breast cancer, inflammatory breast cancer, esophageal cancer, glioma, stomach cancer, lung cancer, and colorectal cancer. In some embodiments, the cancer is pancreatic cancer. In some embodiments, the cancer is a metastatic cancer. In some embodiments, the cancer is metastatic pancreatic cancer. In some embodiments, the cancer is one in which the standard of care for the cancer is associated with the inducement of one or more of the following properties in at least one cancer cell: (a) a mesenchymal cell phenotype (e.g., as characterized by (e.g., increased vimentin expression and/or decreased E-cadherin expression compared to a reference standard (e.g., expression of the marker in a sample from a cancer which has not been treated with the standard of care))); (b) a cancer stem cell phenotype (e.g., as characterized by (e.g., increased expression of one or more embryonically expressed gene (e.g., increased SOX2, OCT-4, and/or Nanog expression compared to a reference standard (e.g., expression of the marker in a sample from a cancer which has not been treated with the standard of care)), increased CD133, CD44, CD24, THY1, EpCAM, ALDH, and/or ABCB5 expression; compared to a reference standard (e.g., expression of the marker in a sample from a cancer which has not been treated with the standard of care))); or (c) an invasive or metastatic cancer cell phenotype (e.g., as characterized by (e.g., increased AKT signaling, increased Rac signaling, increased Rho signaling, increased ROBO signaling, increased expression of one or more gene associated with metastasis; compared to a reference standard (e.g., expression of the marker in a sample from a cancer which has not been treated with the standard of care))). In some embodiments, the standard of care is an antimetabolite (e.g., gemcitabine) at a dosing regimen for that antimetabolite (e.g., gemcitabine). In some embodiments, the standard of care is a platinum based agent (e.g., carboplatin, cisplatin) at a dosing regimen for that platinum based agent (e.g., carboplatin, cisplatin). In some embodiments, the standard of care is a taxane (e.g., docetaxel) at a dosing regimen for that taxane (e.g., docetaxel).

In some embodiments, administration of the anti-cancer treatment (e.g., the chemotherapeutic agent) is associated with the cancer becoming refractory, relapsed, and/or resistant to the anti-cancer treatment (e.g., the chemotherapeutic agent).

Also disclosed herein are methods of treating a cancer in a subject, the method comprising administering a LMWH preparation (e.g., a M402 preparation) described herein, and administering an initial dose of an anti-cancer treatment (e.g., a chemotherapeutic agent), wherein the LMWH preparation (e.g., the M402 preparation) is administered once a week for two weeks; optionally followed by 1 week with no administration of the LMWH nor the anti-cancer treatment; followed by co-administration of the LMWH and the chemotherapeutic agent, wherein the LMWH preparation is administered once a week, for a period of at least 12 weeks (e.g., at least 3 months, 4 months, 5 months, 6 months, 1 year, 2 years, 3 years, 4 years, 5 years, 6 years, 7 years, 8 years, 9 years, 10 years, 1 cycle, 2 cycles, 3 cycles, 4 cycles, 5 cycles, 6 cycles, 7 cycles, 8 cycles, 10 cycles, 20 cycles); and wherein the chemotherapeutic agent is administered once a week for the first 7 weeks including the initial dose of the chemotherapeutic agent; followed by one week with no administration of the chemotherapeutic agent; followed by administered of the chemotherapeutic agent once a week for 3 weeks and no administration for one week of a 28 day cycle, e.g., wherein the chemotherapeutic agent is administered for at least 1, 3, 4, 5, 6, 7, 8, 9, 10, or 20 twenty eight day cycles. In some embodiments, the anti-cancer treatment (e.g., the chemotherapeutic agent) increases wnt/β-catenin signaling.

Also disclosed herein are methods of treating a cancer in a subject, the method comprising administering a LMWH preparation (e.g., a M402 preparation) described herein, and administering an initial dose of a chemotherapeutic agent, wherein the LMWH preparation (e.g., the M402 preparation) is administered prior to the administration of the initial dose of the chemotherapeutic agent; wherein the chemotherapeutic agent is administered once a week for the first 7 weeks; followed by one week with no administration of the chemotherapeutic agent; followed by administration of the chemotherapeutic agent once a week for 3 weeks and no administration for one week of a 28 day cycle, e.g., wherein the chemotherapeutic agent is administered for at least 1, 3, 4, 5, 6, 7, 8, 9, 10, or 20 twenty eight day cycles. In some embodiments, the anti-cancer treatment (e.g., the chemotherapeutic agent) increases wnt/β-catenin signaling. In some embodiments, the cancer is pancreatic cancer (e.g., metastatic pancreatic cancer).

Also disclosed herein are methods of treating a cancer in a subject, the method comprising administering at least one dose of a LMWH preparation (e.g., a M402 preparation) described herein, and administering an initial dose of an anti-cancer treatment (e.g., chemotherapeutic agent), wherein the LMWH preparation (e.g., the M402 preparation) is administered prior to the administration of the initial dose of the anti-cancer treatment (e.g., the chemotherapeutic agent), and wherein the cancer is one in which the standard of care for the cancer is associated with the inducement of one or more of the following properties in at least one cancer cell: (a) a mesenchymal cell phenotype (e.g., as characterized by (e.g., increased vimentin expression and/or decreased E-cadherin expression compared to a reference standard (e.g., expression of the marker in a sample from a cancer which has not been treated with the standard of care))); (b) a cancer stem cell phenotype (e.g., as characterized by (e.g., increased expression of one or more embryonically expressed gene (e.g., increased SOX2, OCT-4, and/or Nanog expression compared to a reference standard (e.g., expression of the marker in a sample from a cancer which has not been treated with the standard of care)), increased CD133, CD44, CD24, THY1, EpCAM, ALDH, and/or ABCB5 expression compared to a reference standard (e.g., expression of the marker in a sample from a cancer which has not been treated with the standard of care))); or (c) an invasive or metastatic cancer cell phenotype (e.g., as characterized by (e.g., increased AKT signaling, increased Rac signaling, increased Rho signaling, increased ROBO signaling, increased expression of one or more gene associated with metastasis; compared to a reference standard (e.g., expression of the marker in a sample from a cancer which has not been treated with the standard of care))). In some embodiments, the standard of care is an antimetabolite (e.g., gemcitabine) at a dosing regimen for that antimetabolite (e.g., gemcitabine). In some embodiments, the standard of care is a platinum based agent (e.g., carboplatin, cisplatin) at a dosing regimen for that platinum based agent (e.g., carboplatin, cisplatin). In some embodiments, the standard of care is a taxane (e.g., docetaxel) at a dosing regimen for that taxane (e.g., docetaxel). In some embodiments, the anti-cancer treatment (e.g., the chemotherapeutic agent) increases wnt/β-catenin signaling.

Also disclosed herein are methods of evaluating or treating a subject having a cancer, wherein the cancer is one in which the standard of care for the cancer is associated with the inducement of one or more of the following properties in at least one cancer cell: (a) a mesenchymal cell phenotype (e.g., as characterized by (e.g., increased vimentin expression and/or decreased E-cadherin expression compared to a reference standard (e.g., expression of the marker in a sample from a cancer which has not been treated with the standard of care))); (b) a cancer stem cell phenotype (e.g., as characterized by (e.g., increased expression of one or more embryonically expressed gene (e.g., increased SOX2, OCT-4, and/or Nanog expression compared to a reference standard (e.g., expression of the marker in a sample from a cancer which has not been treated with the standard of care)), increased CD133, CD44, CD24, THY1, EpCAM, ALDH, and/or ABCB5 expression; compared to a reference standard (e.g., expression of the marker in a sample from a cancer which has not been treated with the standard of care))); or (c) an invasive or metastatic cancer cell phenotype (e.g., as characterized by (e.g., increased AKT signaling, increased Rac signaling, increased Rho signaling, increased ROBO signaling, increased expression of one or more gene associated with metastasis; compared to a reference standard (e.g., expression of the marker in a sample from a cancer which has not been treated with the standard of care))), the method comprising: optionally, acquiring a subject sample; acquiring an evaluation of and/or evaluating the sample for one or more of the following properties: (a) a marker associated with a mesenchymal cell phenotype (e.g., increased vimentin and/or decreased E-cadherin expression compared to a reference standard (e.g., expression of the marker in a sample from a cancer which has not been treated with the standard of care)); (b) a marker associated with a cancer stem cell phenotype (e.g., increased expression of one or more embryonically expressed gene (e.g., increased SOX2, OCT-4, and/or Nanog expression compared to a reference standard), increased CD133, CD44, CD24, THY1, EpCAM, ALDH, and/or ABCB5 expression compared to a reference standard (e.g., expression of the marker in a sample from a cancer which has not been treated with the standard of care)); or (c) a marker associated with an invasive or metastatic cancer phenotype (e.g., increased AKT signaling, increased Rac signaling, increased Rho signaling, increased ROBO signaling, increased expression of one or more gene associated with metastasis compared to a reference standard (e.g., expression of the marker in a sample from a cancer which has not been treated with the standard of care)); and selecting the subject for treatment with a LMWH preparation (e.g., a M402 preparation) described herein, if the subject sample has one or more of the properties; and optionally, administering at least one dose of the LMWH preparation (e.g., the M402 preparation) to the selected subject.

In some embodiments, the standard of care is an antimetabolite (e.g., gemcitabine) at a dosing regimen for that antimetabolite (e.g., gemcitabine). In some embodiments, the standard of care is a platinum based agent (e.g., carboplatin, cisplatin) at a dosing regimen for that platinum based agent (e.g., carboplatin, cisplatin). In some embodiments, the standard of care is a taxane (e.g., docetaxel) at a dosing regimen for that taxane (e.g., docetaxel). In some embodiments, the subject has been treated with an anti-cancer treatment (e.g., a chemotherapeutic agent) which is associated with the inducement of one or more of the following properties in at least one cancer cell: (a) a mesenchymal cell phenotype (e.g., increased vimentin and/or decreased E-cadherin expression compared to a reference standard (e.g., expression of the marker in a sample from a cancer which has not been treated with the anti-cancer treatment)); (b) a cancer stem cell phenotype (e.g., increased expression of one or more embryonically expressed gene (e.g., increased SOX2, OCT-4, and/or Nanog expression compared to a reference standard (e.g., expression of the marker in a sample from a cancer which has not been treated with the anti-cancer treatment)), increased CD133, CD44, CD24, THY1, EpCAM, ALDH, and/or ABCB5 expression compared to a reference standard (e.g., expression of the marker in a sample from a cancer which has not been treated with the anti-cancer treatment)); or (c) an invasive or metastatic cancer phenotype (e.g., increased AKT signaling, increased Rac signaling, increased Rho signaling, increased ROBO signaling, increased expression of one or more gene associated with metastasis compared to a reference standard (e.g., expression of the marker in a sample from a cancer which has not been treated with the anti-cancer treatment)).

In some embodiments, the methods further comprise administering at least one dose of the LMWH preparation (e.g., the M402 preparation) to the selected subject; and administering the anti-cancer agent subsequent to the administration of at least one dose of the LMWH preparation (e.g., the M402 preparation).

In any aspect, embodiment, or method described herein, the LMWH preparation (e.g., the M402 preparation) can comprise a polysaccharide of Formula (I)

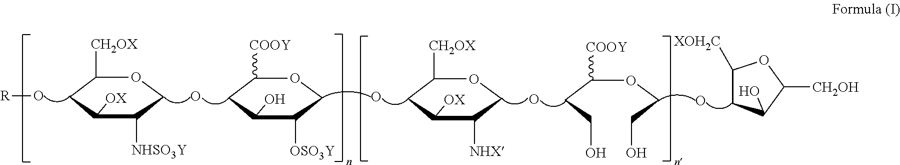

wherein,
each X is independently H or SO₃Y;
each X′ is independently COCH₃ or SO₃Y;
each Y is independently a singularly charged cation such as Na⁺, K⁺, or NH₄⁺;
n is an integer from 5 to 14, e.g., 6 to 12;
n′ is 1, 2 or 3, e.g., 1 or 2; and

### R is

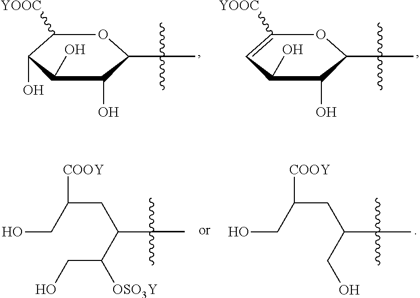

In some embodiments, the compound of Formula (I) is a compound of Formula (Ia)

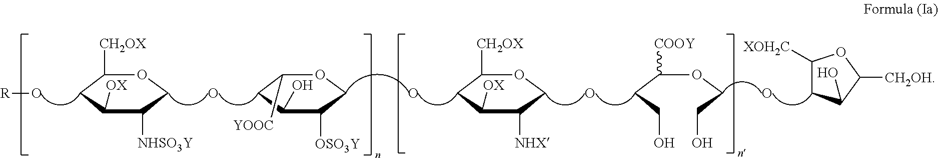

In some embodiments, Y for each occurrence is Na⁺.

In some embodiments, R is

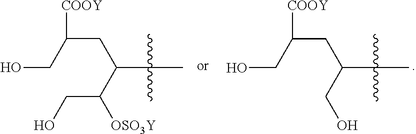

In some embodiments, the LMWH preparation (e.g., the M402 preparation) comprises or consists essentially of:

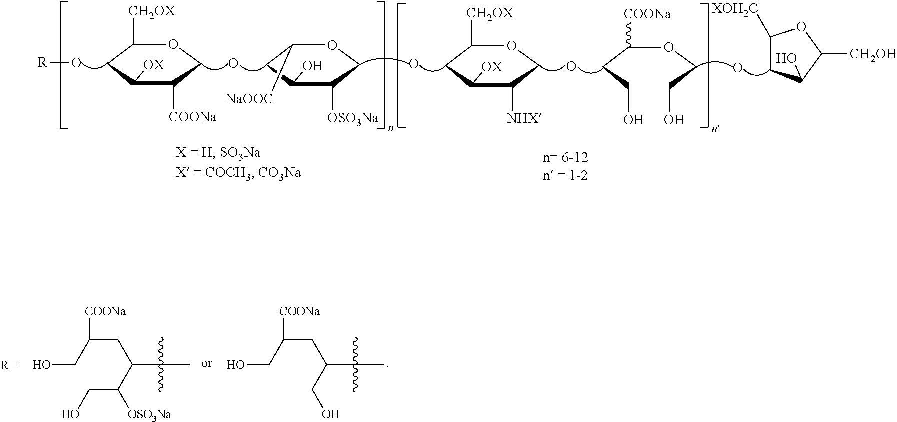

In some embodiments, the LMWH preparation is an M402 preparation.

In some embodiments, the LMWH preparation is

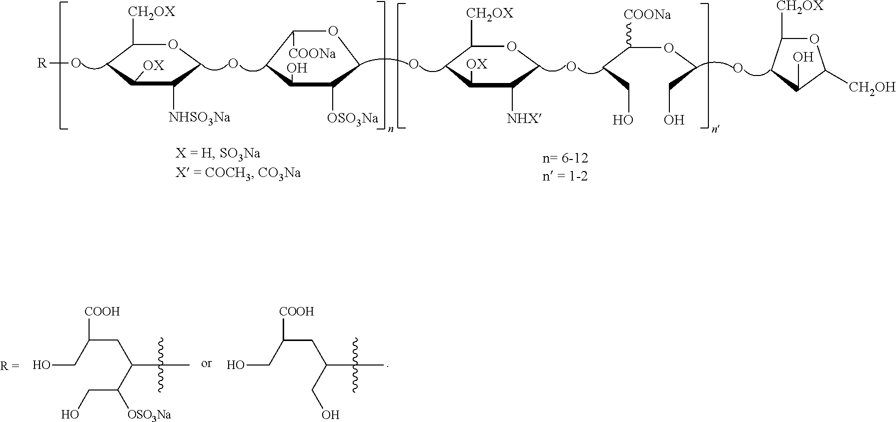

In some embodiments, the LMWH preparation (e.g., the M402 preparation) has a molecular weight distribution such that 10-40% of the oligosaccharides of the preparation have a molecular weight<3000 Da; 45-65% of the oligosaccharides have a molecular weight between 3000-8000 Da, and 15-30% of the oligosaccharides have a molecular weight>8000 Da.

In some embodiments, the LMWH preparation (e.g., the M402 preparation) has the following characteristics:

(a) a weight average chain molecular weight between 3,500 and 8,000 Da;

(b) anti-Xa activity of less than 20 IU/mg and anti-IIa activity of less than 20 IU/mg;

(c) greater than 5% and less than 25% glycol split uronic acid residues; and

(d) the polysaccharide preparation has a molecular weight distribution such that 10-40% of the oligosaccharides of the preparation have a molecular weight<3000 Da; 45-65% of the oligosaccharides have a molecular weight between 3000-8000 Da, and 15-30% of the oligosaccharides have a molecular weight>8000 Da.

### BRIEF DESCRIPTION OF THE FIGURES

FIG. 1A-D demonstrate the applicability of the KPFMC pancreatic cancer mouse model for the recapitulation of human pancreatic ductal adenocarcinoma (PDA). FIG. 1A depicts a macroscopic view of KPFMC mice at the moribund stage clinical endpoint. Individual organs are indicated as labeled. FIG. 1B depicts a microscopic view of KPFMC mice showing the development of pancreatic tumors. Between day 30 and 45 after birth the amount of stroma increases, after which the tumors grow rapidly between day 45 and 51. FIG. 1C is a graph showing the diminished survival of KPFMC mice caused by the rapid growth of the pancreatic tumors. FIG. 1D is H&E staining of a pancreatic tumor from a KPFMC mouse and a human PDA. The staining shows the tumors are comparable.

FIG. 2 is a graph depicting the effect of pretreatment with M402 on survival of KPFMC mice subsequently treated with gemcitabine. KPFMC mice were treated with: Saline (NaCl) or M402 (40 mg/kg) delivered using a subcutaneously (SC) implanted osmotic pump (treatment stated on day 30 after birth). The mice were then treated with NaCl or gemcitabine (50 mg/kg) injected intraperitoneally (IP) starting on day 45. M402 plus gemcitabine combination treatment significantly extended survival in KPFMC mice over saline treatment or gemcitabine monotherapy ((P=0.049 Log-Rank Test, Hazard ratio=(0.21-0.99); 95% confidence interval) M402+gemcitabine group n=16, Saline+gemcitabine n=16, M402+Saline n=9, Saline+saline n=10 and untreated n=9).

FIG. 3A-D demonstrate the effect of gemcitabine treatment on the invasiveness of pancreatic tumors in KPFMC mice. FIG. 3A depicts H&E sections of pancreatic tumors from KPFMC mice at clinical endpoint. The tumors were analyzed microscopically to identify intestinal invasion (upper panel). The lower panel shows the number of mice with intestinal invasion increased upon gemcitabine treatment (100 mg/kg) compared to saline treated mice (lower panel). FIG. 3B is a bar graph depicting the percentage of KPFMC mice showing biliary tract obstructions, indicating invasion increased after gemcitabine treatment (mice sacrificed at clinical endpoint). FIG. 3C depicts phospho-RTK array analysis of KPFMC tumors treated with gemcitabine, showing gemcitabine treatment is associated with an increase in RTK phosphorylation. Expression values are depicted in the heat map. FIG. 3D is a bar graph showing the influence of gemcitabine treatment on the invasive behavior of tumor cells. The cells were analyzed by quantification of double positive E-cadherin and Fsp1 cells in tumor sections of mice treated with saline and gemcitabine until clinical endpoint.

FIG. 4A-C demonstrate the effect of M402 treatment on tumor cell invasion. FIG. 4A depicts a phospho-RTK array showing diminished RTK phosphorylation in pancreatic tumors of mice treated with M402 (40 mg/kg) compared to mice treated with Saline. Expression values are depicted in the heat map. Mice were treated from day 30 after birth until day 51. FIG. 4B is an immunohistochemistry staining of E-cadherin and Vimentin from tumors of KPFMC mice treated with Saline and M402 showing decreased Vimentin expression after treatment with M402. FIG. 4C is an immunofluorescence image of a section from a saline treated KPFMC mouse pancreas co-stained with E-cadherin and Fsp1 showing high magnification view of cells that co-express both the epithelial and mesenchymal marker (left panel). Quantification of E-cadherin and Fsp1 double positive cells indicated a decrease in cells undergoing the transition towards the invasive phenotype in pancreatic tumors treated with M402 (right panel).

FIG. 5A-C demonstrate the effect of pretreatment with M402 on the invasive behavior of tumor cells induced by gemcitabine. FIG. 5A depicts a phospho-RTK array showing inhibition of RTK phosphorylation by M402. Expression values are depicted in the heat map. Color intensities indicate the fold change with M402+gemcitabine combination therapy over M402. FIG. 5B is a bar graph showing the influence of M402 treatment on the invasive behavior of tumor cells induced by gemcitabine. Tumor sections derived from mice treated with saline and gemcitabine until clinical endpoint were analyzed by quantification of cells positive for E-cadherin and Fsp1 in tumor. FIG. 5C is a bar graph showing the quantification of intact Islets of Langerhans (lower panel) from pancreatic tumors after the indicated treatments. (Means±SD are depicted; *P<0.05, NS P>0.05, Student's t test).

FIGS. 6A and 6B depict the effect of gemcitabine treatment on the Wnt/β-catenin signaling pathway. Treatment with gemcitabine increases expression of several members of the Wnt/β-catenin signaling pathway including, e.g., LRH-1, c-JUN, c-Myc, Cyclin D1, TCF1, TCF4, PPARδ, MMP1, CX43, Jun-2, CD44, Oct-4, p14ARF, Wnt, β-catenin, Frizzled, CBP, Blc9, and LEF/TCF.

FIGS. 7A and 7B depict the effect of pretreatment with M402 followed by subsequent gemcitabine/M402 combination treatment on the Wnt/β-catenin signaling pathway. Pretreatment with M402 reduced the gemcitabine induced Wnt/β-catenin signaling, e.g., decreasing expression of several members of the Wnt/β-catenin signaling pathway including, e.g., TGFβ, TGFβR, HPK1, TAK1, CKK, LRP1/5/6, KREMEN, SFRP, CK1/2, PAR1, p53, p14ARF, cadherin, PIN1, CK1, ILK, AKT, GSK3, Axin, PP2A, SOX, REPTIN, APPL, Grouche, LEF/TCF, LRH-1, c-JUN, c-Myc, TCF1, TCF4, PPARδ, MMP1, CX43, Jun-2, CD44, Oct-4, p14ARF, Wnt, β-catenin, Frizzled, CBP, Blc9, and LEF/TCF.

FIG. 8 demonstrates the effect of M402 pretreatment on tumor weight. Pretreatment with M402 followed by combination treatment of M402, gemcitabine (Gemzar), and abraxane significantly reduced primary tumor weight compared to controls.

FIG. 9A-B demonstrate the effect of M402 pretreatment on chemotherapy induced immune cell levels. Gemcitabine and abraxane administration decreased white blood cells (WBCs) (FIG. 9A) and platelet (PLT) counts (FIG. 9B), while pretreatment with M402 followed by combination treatment of M402, gemcitabine (Gemzar), and abraxane rescued the levels of WBCs (FIG. 9A) and PLTs (FIG. 9B).

### DETAILED DESCRIPTION

Anti-cancer treatments (e.g., chemotherapeutic agents, e.g., gemcitabine), can be associated with the development of invasive and/or chemotherapy resistant tumors. As described herein, prior or concurrent administration of LMWHs (e.g., M402) can prevent or ameliorate the development of said tumor phenotype.

### LMWH Preparations

The pharmaceutical compositions described herein include a low molecular weight heparin (LMWH) preparation having at least one chain having a glycol split uronic acid residue (U_{G}) in the preparation.

The disclosed pharmaceutical compositions include glycol split LMWH preparations designed to lack substantial anticoagulant activity while retaining clinically advantageous properties. Properties of the glycol split LMWH preparations include, e.g., lacking substantial anticoagulant activity, e.g., anti-IIa activity less than 50 IU/mg (e.g., anti-IIa activity less than 1 IU/mg), anti-Xa activity less than 50 IU/mg (e.g., anti-Xa activity less than 10 IU/mg), and having anti-metastatic, anti-angiogenic, anti-fibrotic and/or anti-inflammatory activity.

In some embodiments, the LMWH preparation comprises at least one chain having a glycol split uronic acid residue (U_{G}) and, e g., the preparation can lack substantial anticoagulant activity (e.g., preparations of polysaccharides that have reduced anticoagulant activity) but retain activity in other non-coagulation mediated biological processes. For example, these LMWH preparations can have one or more of the following features: 1) anti-Xa activity, e.g., less than 50 IU/mg, 20 IU/mg, 10 IU/mg, 5 IU/mg, 3 IU/mg, 2 IU/mg, 1 IU/mg or less, 2) anti-IIa activity, e.g., less than 50 IU/mg, 20 IU/mg, 10 IU/mg, 5 IU/mg, 3 IU/mg, 2 IU/mg, 1 IU/mg or less and 3) anti-metastatic, anti-angiogenic, anti-fibrotic and/or anti-inflammatory activity. A LMWH preparation provided herein can also have one or more of the following characteristics: the preparation has glycol split uronic acid residues (U_{G}) (e.g., less than 50%, 40%, 30%, 20% glycol split uronic acid residues (U_{G})); the preparation has no more than 3 glycol split uronic acid residues (U_{G}) per polysaccharide chain; the preparation has greater than 40% U_{2S}H_{NS,6S} disaccharide residues present in the chains of the preparation; the degree of desulfation of the preparation is less than 40%; one or more polysaccharide chains in the preparation have a 2,5-anhydromannitol residue at the reducing end. In some preferred embodiments, the weight average molecular weight of the preparation is between 3,500 and 8,000 Da, e.g., between 4,000 and 8,000 Da; and a molecular weight distribution such that 10-50% (e.g., 10-40%, 10-30%, 15-30% or 15-25%) of the oligosaccharides of the preparation have a molecular weight<3000 Da; 40-65% (e.g., 40-60%, 45-65%, 50-65%, or 55-65%) of the oligosaccharides have a molecular weight between 3000-8000 Da, and 5-30% (e.g., 10-30%, 15-30%, 10-25%, or 15-25%) of the oligosaccharides have a molecular weight>8000 Da.

In some embodiments, the LMWH preparation has a weight average molecular weight between 6,000 and 15,000 Da, e.g., between 10,000 and 14,000 Da. In other embodiments, the preparation has a weight average molecular weight between 3,000 and 8,000 Da.

Certain embodiments include a LMWH preparation having the following characteristics: (a) a weight average molecular weight between 3,500 and 8,000 Da, e.g., a weight average molecular weight described herein; (b) anti-Xa activity and/or anti-IIa activity, e.g., less than 50 IU/mg (e.g., anti-Xa activity less than about 40 IU/mg, 30 IU/mg, 20 IU/mg, 15 IU/mg, 10 IU/mg, 5 IU/mg, 4 IU/mg, 3 IU/mg, 2 IU/mg, or 1 IU/mg, and anti-IIa activity less than about 40 IU/mg, 30 IU/mg, 20 IU/mg, 10 IU/mg, 5 IU/mg, 4 IU/mg, 3 IU/mg, 2 IU/mg, or 1 IU/mg); and (c) less than 50% glycol split uronic acid residues (e.g., less than 40%, 30%, 25%, or 20% glycol split uronic acid residues but more than 1%, 5%, 10%, 15%) in the preparation. In some embodiments, the preparation contains between 5% and 50% glycol split uronic acid residues (e.g., between 5% and 40%, 5% and 30%, 10% and 50%, 10% and 40%, 10% and 30%, or 10 and 20% glycol split uronic acid residues). Preferably, the preparation has a molecular weight distribution such that 10-50% (e.g., 10-40%, 10-30%, 15-30% or 15-25%) of the oligosaccharides of the preparation have a molecular weight<3000 Da; 40-65% (e.g., 40-60%, 45-65%, 50-65%, or 55-65%) of the oligosaccharides have a molecular weight between 3000-8000 Da, and 5-30% (e.g., 10-30%, 15-30%, 10-25%, or 15-25%) of the oligosaccharides have a molecular weight>8000 Da.

Certain embodiments include a LMWH preparation having the following characteristics: (a) a weight average chain molecular weight between 3,500 and 8,000 Da; (b) anti-Xa activity of less than 20 IU/mg and anti-IIa activity of less than 20 IU/mg; and (c) greater than 5% and less than 25%, e.g., less than 20, less than 10, glycol split uronic acid residues.

Certain embodiments include a LMWH preparation having the following characteristics: (a) a weight average chain molecular weight between 3,500 and 8,000 Da; (b) anti-Xa activity of less than 20 IU/mg and anti-IIa activity of less than 20 IU/mg; and (c) greater than 5% and less than 25%, e.g., less than 20, less than 10, glycol split uronic acid residues; wherein the preparation has polysaccharide chains of the preparation having greater than 40%, e.g., greater than 50%, 60%, 70%, U_{2S}H_{NS,6S} disaccharide residues within the chains of the preparation.

In some embodiments, the LMWH preparation has one or more chains having a glycol split uronic acid residue and each polysaccharide chain of the preparation having no more than 3, e.g., no more than 2, no more than 1, glycol split uronic acid residues (U_{G}). In some embodiments, the LMWH preparation has one or more chain having a glycol split uronic acid residue and each polysaccharide chain of the preparation having no more than 2 glycol split uronic acid residues (U_{G}). In some embodiments, the LMWH preparation has one or more chains having a glycol split uronic acid residue and each polysaccharide chain of the preparation having no more than 1 glycol split uronic acid residues (U_{G}).

In some embodiments, the LMWH preparation has the following characteristics: (a) a weight average chain molecular weight between 3,500 and 8,000 Da; (b) anti-Xa activity of less than 20 IU/mg and anti-IIa activity of 10 IU/mg or less (e.g., 5 IU/mg, 2 IU/mg, 1 IU/mg); (c) greater than 5% and less than 25% glycol split uronic acid residues; and (d) the polysaccharide preparation has a molecular weight distribution such that 10-40% of the oligosaccharides of the preparation have a molecular weight<3000 Da; 45-65% of the oligosaccharides have a molecular weight between 3000-8000 Da, and 15-30% of the oligosaccharides have a molecular weight>8000 Da.

In some embodiments, the LMWH preparation comprises a polysaccharide of Formula (I)

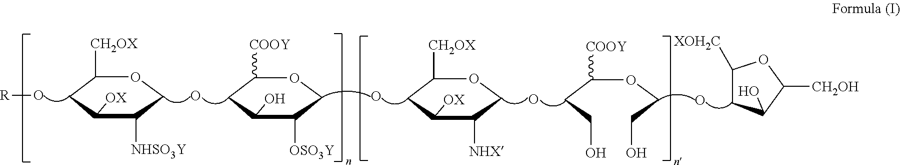

wherein,
each X is independently H or SO₃Y;
each X′ is independently COCH₃ or SO₃Y;
each Y is independently a singularly charged cation such as Na⁺, K⁺, or NH₄⁺;
n is an integer from 5 to 14, e.g., 6 to 12;
n′ is 1, 2 or 3, e.g., 1 or 2; and

### R is

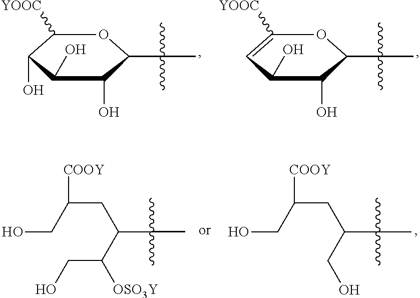

each Y is independently a singularly charged cation such as Na⁺, K⁺, or NH₄⁺.

In some embodiments, the polysaccharide of Formula (I) is a polysaccharide of Formula (Ia)

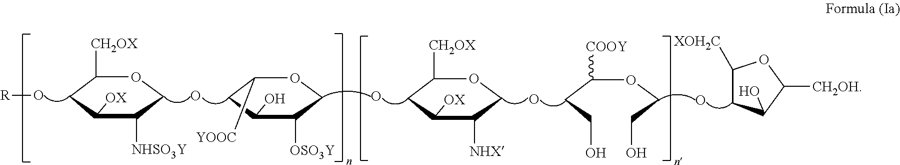

In some embodiments, Y for each occurrence is Na⁺.

In some embodiments, R is,

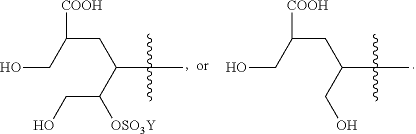

In some embodiments, the polysaccharide of Formula (I) is a polysaccharide of Formula (Ib)

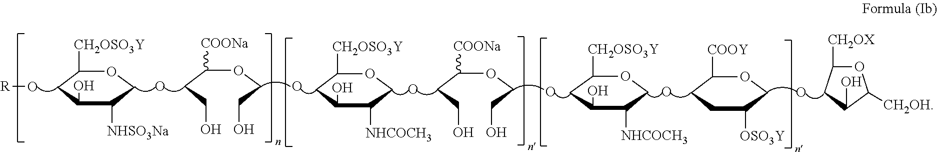

In some embodiments, Y for each occurrence is Na⁺.

In some embodiments, R is,

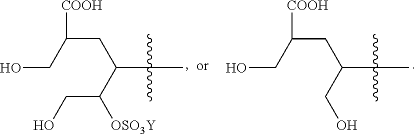

In some embodiments, the preparation consists essentially of polysaccharides having the structure of Formula (I) or Formula (Ia) or Formula (Ib). In some embodiments, the preparation consists of polysaccharides having the structure of Formula (I) or Formula (Ia) or Formula (Ib).

In some embodiments, at least about 20% (e.g., at least about 30%, at least about 40%, at least about 50%, at least about 60%, at least about 70%, at least about 80%, at least about 90%, at least about 95%, at least about 98%, or at least about 99%) of the polysaccharides in the preparation have the structure of Formula (I) or Formula (Ia).

Preferably, the preparation has anti-Xa activity of less than 50 IU/mg, 40 IU/mg, 30 IU/mg, 20 IU/mg or 10 IU/mg but greater than 0.5 IU/mg, 1 IU/mg and/or anti-IIa activity of less than 50 IU/mg, 40 IU/mg, 30 IU/mg, 20 IU/mg or 10 IU/mg but greater than 0.5 IU/mg, 1 IU/mg. In some embodiments, the preparation has a weight average chain molecular weight between 3,500 and 8,000 Da, e.g., between 4,000 and 8000 Da, 4,500 and 8,000 Da, 4,700 and 8,000 Da and 5,000 and 8,000 Da. In some embodiments, the preparation has a molecular weight distribution such that 10-50% (e.g., 10-40%, 10-30%, 15-30% or 15-25%) of the oligosaccharides of the preparation have a molecular weight<3000 Da; 40-65% (e.g., 40-60%, 45-65%, 50-65%, or 55-65%) of the oligosaccharides have a molecular weight between 3000-8000 Da, and 5-30% (e.g., 10-30%, 15-30%, 10-25%, or 15-25%) of the oligosaccharides have a molecular weight>8000 Da. The LMWH preparations described herein (e.g., described above) can also be a pharmaceutically acceptable salt of any of the LMWH preparations described herein.

Any of the preparations described herein, e.g., described above, can have other properties. E.g., one of the above described preparations can further have one or more of the functional or structural properties set out below:

the preparation or pharmaceutical preparation has a molecular weight distribution such that 10-50% (e.g., 10-40%, 10-30%, 15-30% or 15-25%) of the oligosaccharides of the preparation have a molecular weight<3000 Da; 40-65% (e.g., 40-60%, 45-65%, 50-65%, or 55-65%) of the oligosaccharides have a molecular weight between 3000-8000 Da, and 5-30% (e.g., 10-30%, 15-30%, 10-25%, or 15-25%) of the oligosaccharides have a molecular weight>8000 Da;

the preparation has a polydispersity of about 1.2 to 1.7 (e.g., about 1.3 to 1.7, 1.4 to 1.6, or 1.3 to 1.6);

the preparation has a polydispersity of about 1.2 to 1.8 (e.g., about 1.3 to 1.8, 1.4 to 1.7, or 1.3 to 1.7);

the preparation or preparation has a sodium content less than 30%, 25%, 20%, 15%, 10%. In one embodiment, the preparation or preparation comprises: less than 20 ppm, 15 ppm, 10 ppm, 5 ppm iodine; less than 30%, 25%, 20%, 15%, 10% sulfur; less than 50, 40, 30, 20, 15 ppm boron;

the preparation or preparation has anti-metastatic activity;

the preparation or preparation binds specifically to or inhibits an activity of one or more of: VEGF, FGF, SDF-1-α, HB-EGF, heparanase, SCF, sonic hedgehog, osteopontin, osteopontegerin or P-selectin.

In some embodiments, the LMWH preparation is an M402 preparation. An “M402 preparation” refers to a LMWH preparation that consists essentially of:

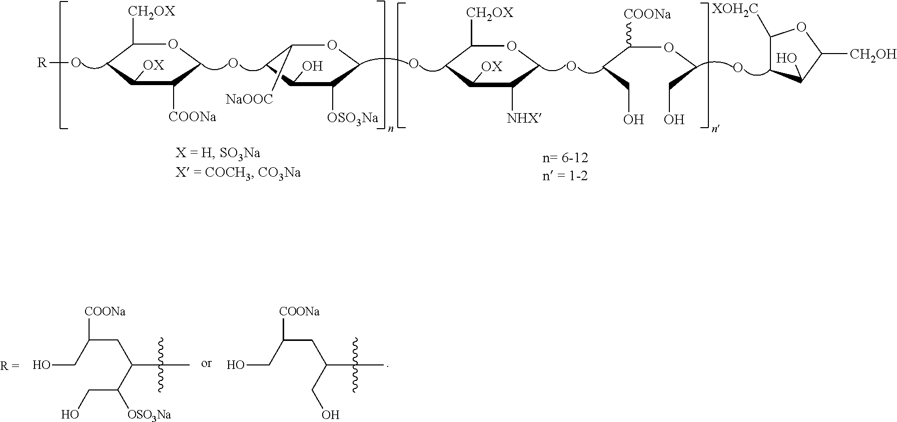

Which is also represented as:

C_{[12(n+n′)+6]H[(14n+7n′)+10]N(n++n′)}Na_{3n+n′}O_{[16n+10n′)+5]}S_{2N}RX_{[2(n+n′)+1]}X′_{n′}

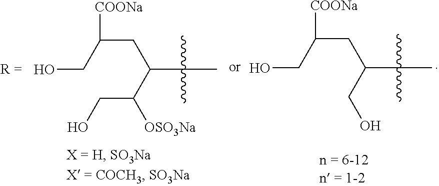

In some embodiments, the M402 preparation is Necuparinol.

The pharmaceutical compositions described herein can have a shelf life of at least 30 days, e.g., at least two months, at least three months, at least six months, at least nine months, twelve months, or at least eighteen months).

Any composition or preparation described herein can be manufactured using good manufacturing practices (GMP) as defined by the U.S. Food and Drug Administration (21 CFR Part 110).

Anti-IIa Activity

LMWH preparations are disclosed herein that provide substantially reduced anti-IIa activity, e.g., anti-IIa activity of about less than about 50 IU/mg, less than about 40 IU/mg, 30 IU/mg, 20 IU/mg, 15 IU/mg, 10 IU/mg, 5 IU/mg, 4 IU/mg, 3 IU/mg, 2 IU/mg, 1 IU/mg or less; or from about 0 to 50 IU/mg, about 0 to 40 IU/mg, about 0 to 30 IU/mg, about 0 to 25 IU/mg, about 0 to 20 IU/mg, about 0 to 10 IU/mg, about 0 to 5 IU/mg, about 5 to 10 IU/mg, about 5 to 15 IU/mg, or about 5 to 20 IU/mg). Anti-IIa activity is calculated in International Units of anti-IIa activity per milligram using statistical methods for parallel line assays. The anti-IIa activity levels described herein are measured using the following principle.

Polysaccharide(PS)+ATIII→[PS.ATIII]

IIa

PS.ATIII→[PS.ATIII.IIa]+IIa(Excess)

IIa(Excess)+Substrate→Peptide+pNA(measured spectrophotometrically)

Anti-factor IIa activity is determined by the sample potentiating effect on antithrombin (ATIII) in the inhibition of thrombin. Thrombin excess can be indirectly spectrophotometrically measured. The anti-factor IIa activity can be measured, e.g., on a Diagnostica Stago analyzer or on an ACL Futura3 Coagulation system, with reagents from Chromogenix (S-2238 substrate, Thrombin (53 nkat/vial), and Antithrombin), or on any equivalent system. Analyzer response is calibrated using the 2nd International Standard for Low Molecular Weight Heparin.

Anti-Xa Activity

In some embodiments, a LMWH preparation provided herein has anti-Xa activity of less than about 50 IU/mg, less than about 40 IU/mg, 30 IU/mg, 20 IU/mg, 15 IU/mg, 10 IU/mg, 5 IU/mg, 4 IU/mg, 3 IU/mg, 2 IU/mg, 1 IU/mg or less, or about 0 to 50 IU/mg, e.g., 50 IU/mg, 40 IU/mg, 30 IU/mg, 20 IU/mg, 15 IU/mg, 10 IU/mg, 5 IU/mg, 4 IU/mg, 3 IU/mg, 2 IU/mg or 1 IU/mg; or from about 0 to 50 IU/mg, about 0 to 40 IU/mg, about 0 to 30 IU/mg, about 0 to 25 IU/mg, about 0 to 20 IU/mg, about 0 to 10 IU/mg, about 0 to 5 IU/mg, about 5 to 10 IU/mg, about 5 to 15 IU/mg, or about 5 to 20 IU/mg). In some embodiments, a LMWH preparation provided herein has anti-Xa activity of about 2 IU/mg. Anti-Xa activity of a preparation is calculated in International Units of anti-factor Xa activity per milligram using statistical methods for parallel line assays. The anti-factor Xa activity of preparations described herein is measured using the following principle:

PS+ATIII→[PS.ATIII]

FXa

PS.ATIII→[PS.ATIII.FXa]+FXa(Excess)

FXa(Excess)+Substrate→Peptide+pNA(measured spectrophotometrically)

The anti-factor Xa activity is determined by the sample potentiating effect on antithrombin (ATIII) in the inhibition of activated Factor Xa (FXa). Factor Xa excess can be indirectly spectrophotometrically measured. Anti-factor Xa activity can be measured, e.g., on a Diagnostica Stago analyzer with the Stachrom® Heparin Test kit, on an ACL Futura3 Coagulation system with the Coatest® Heparin Kit from Chromogenix, or on any equivalent system. Analyzer response can be calibrated using the NIBSC International Standard for Low Molecular Weight Heparin.

Molecular Weight and Chain Length

LMWH preparations included in the pharmaceutical compositions can have a weight average molecular weight described herein.

“Weight average molecular weight” as used herein refers to the weight average in daltons of chains of uronic acid/hexosamine disaccharide repeats. The weight average molecular weight (M_{w}) is calculated from the following equation: M_{w}=Σ(cᵢmᵢ)/Σcᵢ. The variable cᵢ is the concentration of the polymer in slice i and mᵢ is the molecular weight of the polymer in slice i. The summations are taken over a chromatographic peak, which contains many slices of data. A slice of data can be pictured as a vertical line on a plot of chromatographic peak versus time. The elution peak can therefore be divided into many slices. The weight average molecular weight calculation is average dependent on the summation of all slices of the concentration and molecular weight. The weight average molar weight can be measured, e.g., using the Wyatt Astra software or any appropriate software. The weight average molecular weights described herein are determined by high liquid chromatography with two columns in series, for example a TSK G3000 SWXL and a G2000 SWXL, coupled with a UV or multi angle light scattering (MALS) detector and a refractometric detector in series. The eluent used is a 0.2 M sodium sulfate, pH 5.0, and a flow rate of 0.5 mL/min.

A determination of whether a LMWH preparation includes chains of sufficient chain length can be made, for example, by determining the average chain length of the chains in the preparation and/or by determining the weight average molecular weight of chains within the preparation. For example, when weight average molecular weight of a preparation is determined, a weight average molecular weight of about 3500 to 8000 Da, about 4000 to 8000 Da, about 4200 to 8000, or about 4500 to 8000 Da, indicates that a significant number of chains in the LMWH preparation are of a chain length described herein, e.g., for M402, n+n′ has an average chain length of 7 to 14.

“Average chain length” as used herein refers to the average chain length of uronic acid/hexosamine disaccharide repeats that occur within a chain. Average chain length is determined by dividing the number average molecular weight (Mn) by the number average molecular weight for a disaccharide (500 Da).

Molecular Weight Distribution

The molecular weight distribution of a LMWH preparation described herein can be determined by known methods.

In some embodiments, a LMWH preparation described herein has a molecular weight distribution such that 10-50% (e.g., 10-40%, 10-30%, 15-30% or 15-25%) of the oligosaccharides of the preparation have a molecular weight<3000 Da; 40-65% (e.g., 40-60%, 45-65%, 50-65%, or 55-65%) of the oligosaccharides have a molecular weight between 3000-8000 Da, and 5-30% (e.g., 10-30%, 15-30%, 10-25%, or 15-25%) of the oligosaccharides have a molecular weight>8000 Da. In certain embodiments, a LMWH preparation described herein has a molecular weight distribution such that 10-40% of the oligosaccharides of the preparation have a molecular weight<3000 Da; 45-65% of the oligosaccharides have a molecular weight between 3000-8000 Da, and 15-30% of the oligosaccharides have a molecular weight>8000 Da.

Glycol Split Uronic Acids

A LMWH preparation described herein can include an opening of a glycoside ring, conventionally called reduction-oxidation (RO) derivative. In these preparations, one or more glycoside rings having vicinyl diols that are opened, e.g., at the bond between C2 and C3, by means of an oxidation action, followed by a reduction. The compounds referred to herein will also be called “Glycol Split” derivatives.

In a further embodiment of the invention described herein, the glycol split residues lend themselves to the subsequent functionalization. Therefore, polysaccharides of the preparation may also bear equal or different groups, in place of the primary hydroxy groups deriving from glycol split, for example, aldehyde groups, methoxy groups, or oligosaccharide or peptide groups, ranging from a single saccharide or amino acid to more than one unit of length, e.g., 2 or 3 units.

In some embodiments, fewer than 50% of the total uronic acid residues are glycol split uronic acid residues (e.g., less than 40%, 30%, 25%, or 20% of the total uronic acid residues are glycol split uronic acid residues, e.g., but more than 1%, 2%, 3%, 5% of the total uronic acid residues are glycol split uronic acid residues).

Reducing End Structures

In some instances, at least about 50% of the chains in a LMWH preparation described herein have a modified reducing end structure such as a 2,5-anhydromannose residue or a 2,5-anhydromannose that has been reduced to form an alcohol. In some embodiments, at least about 55%, 60%, 65%, 70%, 75%, 80%, 85%, 90%, or 95% of the chains in the preparation have a modified reducing end structure, such that the reducing end includes a 2,5-anhydromannose residue or a 2,5-anhydromannitol.

Non-Reducing End Structures

In some embodiments, at least about 55%, 60%, 65%, 70%, 75%, 80%, 85%, 90%, 95%, or 100% of the chains of a LMWH preparation described herein have a uronic acid at the non-reducing end. In some embodiments, at least about 55%, 60%, 65%, 70%, 75%, 80%, 85%, 90%, 95%, or 100% of the chains of a LMWH preparation described herein have a non native uronic acid at the non-reducing end. In some embodiments, at least about 55%, 60%, 65%, 70%, 75%, 80%, 85%, 90%, 95%, or 100% of the chains of a LMWH preparation described herein have a glycol split uronic acid at the non-reducing end. In some embodiments, the non reducing end of chains of the LMWH preparation has one or more the following structure:

is

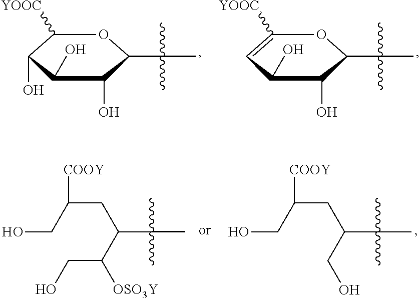

and combinations thereof,
wherein each Y is independently a singularly charged cation such as Na⁺, K⁺, or NH₄⁺.

In one embodiment, the non-reducing end of the chains of the LMWH preparation have the following structures:

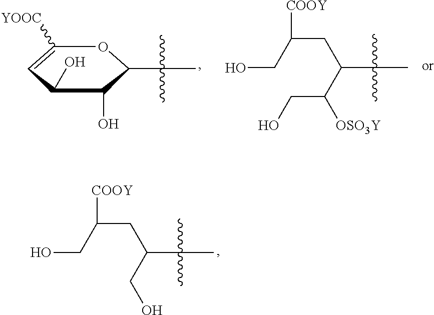

and combinations thereof, wherein each Y is independently a singularly charged cation such as Na⁺, K⁺, or NH₄⁺.

In one embodiment, the non-reducing end of the chains of the LMWH preparation have the following structures:

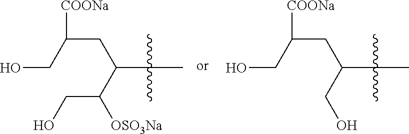

and combinations thereof.

Polydispersity

The polydispersity of LMWH preparations provided herein is about 2 or less, e.g., 1.8, 1.7 or less, e.g., about 1.7 or 1.6 to 1.2, about 1.4-1.5, and numbers in between.

The term “polydisperse” or “polydispersity” refers to the weight average molecular weight of a preparation (Mw) divided by the number average molecular weight (Mn). The number average molecular weight (Mn) is calculated from the following equation: Mn=Σci/(Σci/mi). The variable ci is the concentration of the polysaccharide in slice i and Mi is the molecular weight of the polysaccharide in slice i. The summations are taken over a chromatographic peak, which contains many slices of data. A slice of data can be pictured as a vertical line on a plot of chromatographic peak versus time. The elution peak can therefore be divided into many slices. The number average molecular weight is a calculation dependent on the molecular weight and concentration at each slice of data. Methods of determining weight average molecular weight are described above, and were used to determine polydispersity as well.

### Methods of Making LMWH Preparations

The LMWH preparation can be made, e.g., by known methods. In some embodiments, a LMWH preparation lacking substantial anticoagulant activity can be made by a method that includes providing a precursor LMWH preparation having a weight average molecular weight of greater than 7000 Da or a chain length of greater than 7 to 18 disaccharides, and processing the precursor LMWH preparation (e.g., by enzymatic or chemical depolymerization, e.g., by nitrous acid depolymerization) to obtain a LMWH preparation having a weight average molecular weight of about 3000 to 8000 Da or an average chain length of about 7 to 16 disaccharides. For example, the precursor LMWH preparation can be unfractionated heparin.

The precursor LMWH preparation can be processed by a method comprising depolymerization (e.g., by nitrous acid treatment, hydrolysis, or enzymatic depolymerization) followed by a glycol split reaction. Nitrous acid depolymerization can be accomplished, e.g., by treating the precursor LMWH preparation (e.g., UFH) with nitrous acid (e.g., about 0.02 to 0.04 M nitrous acid) at a pH of about 2 to 4 for a specified period of time (e.g., about 1 to 5 hours) at a temperature of about 10 to 30° C. The glycol split reaction involves periodate oxidation using periodate (e.g., about 0.05 M to 0.2 M sodium periodate) for about 10 to 20 hours at a temperature of about 0 to 10° C. In some embodiments, residual impurities such as salts or diethylene glycol (DEG) can be subsequently removed by a chromatographic method, e.g. gel filtration chromatography. Optionally, the oxidized preparation is then reduced by treatment with a reducing agent (e.g., about 0.5 to 2.0% (w/v) sodium borohydride) for about 0.5 to 3 hours at a pH of about 6.0 to 7.0 and a temperature of about 0 to 10° C.

A precursor LMWH preparation can be processed using enzymatic digestion, chemical digestion or combinations thereof. Examples of chemical digestion include oxidative depolymerization, e.g., with H₂O₂ or Cu⁺ and H₂O₂, deaminative cleavage, e.g., with isoamyl nitrite or nitrous acid, β-eliminative cleavage, e.g., with benzyl ester, and/or by alkaline treatment. Enzymatic digestion can include the use of one or more heparin degrading enzymes. For example, the heparin degrading enzyme(s) can be, e.g., one or more heparanase, heparin lyase, heparan sulfate glycoaminoglycan (HSGAG) lyase, a lyase described as a glycoaminoglycan (GAG) lyase that can also degrade heparin. In some embodiments, the enzyme cleaves at one or more glycosidic linkages of unsulfated uronic acids.

Anti-Cancer Treatments and Modulation of Wnt/β-Catenin Signaling

Described herein are anti-cancer treatments (e.g., chemotherapeutic agents, radiation) that modulate (e.g., increase) Wnt/β-catenin signaling. Wnt/β-catenin signaling can refer to the canonical or non-canonical Wnt/β-catenin signaling pathway. An anti-cancer treatment that increases Wnt/β-catenin signaling can be any anti-cancer treatment that increases expression of, activity of, or a function of, a gene and/or a protein involved in or associated with the Wnt/β-catenin signaling pathway, e.g., TGFβ, TGFβR, HPK1, TAK1, CKK, LRP1/5/6, KREMEN, SFRP, CK1/2, PAR1, p53, p14ARF, cadherin, PIN1, CK1, ILK, AKT, GSK3, Axin, PP2A, SOX, REPTIN, APPL, Grouche, LEF/TCF, LRH-1, c-JUN, c-Myc, TCF1, TCF4, PPARδ, MMP1, CX43, Jun-2, CD44, Oct-4, p14ARF, Wnt, β-catenin, Frizzled, CBP, Blc9, or Cyclin D1.

Chemotherapeutic Agents

In some embodiments, the anti-cancer treatment that modulates (e.g., increases) Wnt/β-catenin signaling is a chemotherapeutic agent (e.g., known in the art or described herein). Exemplary chemotherapeutic agents include but are not limited to an antimetabolite (e.g., gemcitabine, 5-flurouracil), a platinum based agent (e.g., carboplatin, cisplatin), or a taxane (e.g., docetaxel).

Radiation

The anti-cancer treatment can be radiation therapy.

### Administration

The LMWH (e.g., M402) pharmaceutical composition or preparation (e.g., as described herein) can be administered to the subject, in accord with known methods, such as intravenous administration as a bolus or by continuous infusion over a period of time, by intramuscular, intraperitoneal, intracerobrospinal, subcutaneous, intra-articular, intrasynovial, and intrathecal routes. In some embodiments, the LMWH (e.g., M402) preparation is administered to the subject by intravenous administration, e.g., as a bolus or by continuous infusion over a period of time. In an embodiment, the LMWH (e.g., M402) preparation is administered to the subject by subcutaneous (i.e. beneath the skin) administration. For such purposes, the preparation may be injected using a syringe. However, other devices for administration of the preparation are available such as injection devices (e.g. the Inject-Ease™ and Genject™ devices); injector pens (such as the GenPen™); needleless devices (e.g. MediJector™ and BioJector™); and subcutaneous patch delivery systems.

The anti-cancer treatment can be administered to the subject, in accord with known methods, such as intravenous administration as a bolus or by continuous infusion over a period of time, by intramuscular, intraperitoneal, intracerobrospinal, subcutaneous, intra-articular, intrasynovial, and intrathecal routes.

### Dosage and Dosage Regimen

LMWH Dosage

The appropriate dosage (e.g., therapeutically effective amount) of the LMWH preparation will depend, for example, on the condition to be treated, the severity and course of the condition, previous therapy, the patient's clinical history, the type of LMWH used, and the discretion of the attending physician.

The LMWH preparation (e.g., the M402 preparation), can be administered at a dose from 1 to 40 mg/kg (e.g., 1-20 mg/kg, 1-10 mg/kg, 2-9 mg/kg, 3-8 mg/kg). The LMWH preparation (e.g., the M402 preparation), can be administered at a dose of 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg, 20 mg/kg or 40 mg/kg. In some embodiments, the LMWH preparation (e.g., the M402 preparation), is administered at a dose of 1 mg/kg. In some embodiments, the LMWH preparation (e.g., the M402 preparation), is administered at a dose of 2 mg/kg. In some embodiments, the LMWH preparation (e.g., the M402 preparation), is administered at a dose of 3 mg/kg. In some embodiments, the LMWH preparation (e.g., the M402 preparation), is administered at a dose of 4 mg/kg. In some embodiments, the LMWH preparation (e.g., the M402 preparation), is administered at a dose of 5 mg/kg. In some embodiments, the LMWH preparation (e.g., the M402 preparation), is administered at a dose of 6 mg/kg. In some embodiments, the LMWH preparation (e.g., the M402 preparation), is administered at a dose of 8 mg/kg. In some embodiments, the LMWH preparation (e.g., the M402 preparation), is administered at a dose of 10 mg/kg. In some embodiments, the LMWH preparation (e.g., the M402 preparation), is administered at a dose of 15 mg/kg. In some embodiments, the LMWH preparation (e.g., the M402 preparation), is administered at a dose of 20 mg/kg. In some embodiments, the LMWH preparation (e.g., the M402 preparation), is administered at a dose of 25 mg/kg. In some embodiments, the LMWH preparation (e.g., the M402 preparation), is administered at a dose of 30 mg/kg. In some embodiments, the LMWH preparation (e.g., the M402 preparation), is administered at a dose of 40 mg/kg.

Anti-Cancer Treatment Dosage

The appropriate dosage (“therapeutically effective amount”) of the anti-cancer treatment will depend, for example, on the condition to be treated, the severity and course of the condition, previous therapy, the patient's clinical history, the type of anti-cancer treatment used, and the discretion of the attending physician. The anti-cancer treatment can be administered at a dose known in the art for the anti-cancer treatment. The anti-cancer treatment can be administered at a dose that is the standard of care for the specific anti-cancer agent when treating a specific condition, e.g., the standard of care at the time the cancer treatment is to be administered. In some embodiments, the anti-cancer treatment is a chemotherapeutic agent administered at an amount known in the art for the chemotherapeutic agent.

In some embodiments, the anti-cancer treatment is gemcitabine administered at a dose known in the art for gemcitabine. In some embodiments, the anti-cancer treatment is gemcitabine administered at a dose that is the standard of care for treating a specific condition, e.g., pancreatic cancer, e.g., the standard of care at the time gemcitabine is to be administered. In certain embodiments, the anti-cancer treatment is gemcitabine administered at a dose between 500 mg/m² and 1500 mg/m². In certain embodiments, the anti-cancer treatment is gemcitabine administered at a dose of about 1000 mg/m² or 1250 mg/m². In certain embodiments, the anti-cancer treatment is gemcitabine and is administered at a dose of about 1000 mg/m².

In some embodiments, the anti-cancer treatment is abraxane administered at a dose known in the art for abraxane. In certain embodiments, the anti-cancer treatment is abraxane administered at a dose between 50 mg/m² and 300 mg/m². In certain embodiments, the anti-cancer treatment is abraxane administered at a dose of about 260 mg/m², 100 mg/m², or 125 mg/m². In certain embodiments, the anti-cancer treatment is abraxane and is administered at a dose of about 1000 mg/m².

Dosing Regimen

The LMWH preparation (e.g., the M402 preparation) can be administered at least 1 hour (e.g., at least 2 hours, at least 3 hours, at least 4 hours, at least 5 hours, at least 6 hours, at least 12 hours, 24 hours, 37 hours, 48 hours) prior to the administration of the initial dose of the anti-cancer treatment (e.g., the chemotherapeutic agent). The LMWH preparation (e.g., the M402 preparation), can be administered at least 1 day (e.g., at least 2 days, 3 days, 4 days, 5 days, 6 days, 7 days) prior to the administration of the initial dose of the anti-cancer treatment (e.g., chemotherapeutic agent). The LMWH preparation (e.g., the M402 preparation), can be administered at least 1 week (e.g., at least 2 weeks, 3 weeks, 4 weeks, 5 weeks, 6 weeks) prior to the administration of the initial dose of the anti-cancer treatment (e.g., chemotherapeutic agent). The LMWH preparation (e.g., the M402 preparation), can be administered at least 1 month (e.g., at least 2 months, 3 months, 4 months, 5 months, 6 months) prior to the administration of the initial dose of the anti-cancer treatment (e.g., chemotherapeutic agent). The LMWH preparation (e.g., the M402 preparation), can be administered within 6 hours or less (e.g., 5 hours or less; 4 hours or less; 3 hours or less; 2 hours or less; 1 hour or less) of the administration of the initial dose of the anti-cancer treatment (e.g., chemotherapeutic agent).

More than one dose (e.g., 2, 3, 4, 5, 6, 7, 8, 9, 10 or more doses) of the LMWH preparation (e.g., the M402 preparation), can be administered to the subject.

The LMWH preparation (e.g., the M402 preparation) can be administered daily (e.g., once daily, twice daily), weekly, every other week, every three weeks, monthly, every other month, or every three months. The LMWH preparation (e.g., the M402 preparation), can be administered daily, e.g., once daily. The LMWH preparation (e.g., the M402 preparation), can be administered weekly, e.g., once weekly. The LMWH preparation (e.g., the M402 preparation), can be administered monthly, e.g., once monthly. The LMWH preparation (e.g., the M402 preparation), can be administered every other month monthly, e.g., once every other monthly. The LMWH preparation (e.g., the M402 preparation), can be administered every three months, e.g., once every three months.

The LMWH preparation (e.g., the M402 preparation) can be administered daily (e.g., once daily, twice daily), weekly, every other week, every three weeks, monthly, every other month, or every three months for a period of at least 1 week (e.g., at least 2 weeks, 3 weeks, 4 weeks, 5 weeks, 6 weeks, 7 weeks, 1 month, 2 months, 3 months, 4 months, 5 months, 6 months, 1 year). The LMWH preparation (e.g., the M402 preparation), can be administered daily, e.g., once daily, for a period of at least 1 week (e.g., at least 2 weeks, 3 weeks, 4 weeks, 5 weeks, 6 weeks, 7 weeks, 1 month, 2 months, 3 months, 4 months, 5 months, 6 months, 1 year). The LMWH preparation (e.g., the M402 preparation), can be administered weekly, e.g., once weekly, for a period of at least 1 week (e.g., at least 2 weeks, 3 weeks, 4 weeks, 5 weeks, 6 weeks, 7 weeks, 1 month, 2 months, 3 months, 4 months, 5 months, 6 months, 1 year). The LMWH preparation (e.g., the M402 preparation), can be administered according to a dosing regimen for M402.

More than one dose (e.g., 2, 3, 4, 5, 6, 7, 8, 9, 10 or more doses) of the anti-cancer treatment (e.g., the chemotherapeutic agent), can be administered to the subject.

The anti-cancer treatment (e.g., the chemotherapeutic agent) can be administered daily (e.g., once daily, twice daily), weekly, every other week, every three weeks, monthly, every other month, or every three months. The anti-cancer treatment (e.g., the chemotherapeutic agent), can be administered daily, e.g., once daily. The anti-cancer treatment (e.g., the chemotherapeutic agent), can be administered weekly, e.g., once weekly. The anti-cancer treatment (e.g., the chemotherapeutic agent), can be administered monthly, e.g., once monthly. The anti-cancer treatment (e.g., the chemotherapeutic agent), can be administered every other month y, e.g., once every other month. The anti-cancer treatment (e.g., the chemotherapeutic agent), can be administered every three months, e.g., once every three months.

The anti-cancer treatment (e.g., the chemotherapeutic agent) can be administered daily (e.g., once daily, twice daily), weekly, every other week, every three weeks, monthly, every other month, or every three months for a period of at least 1 week (e.g., at least 2 weeks, 3 weeks, 4 weeks, 5 weeks, 6 weeks, 7 weeks, 1 month, 2 months, 3 months, 4 months, 5 months, 6 months, 1 year), as appropriate. The anti-cancer treatment (e.g., the chemotherapeutic agent), can be administered daily, e.g., once daily, for a period of at least 1 week (e.g., at least 2 weeks, 3 weeks, 4 weeks, 5 weeks, 6 weeks, 7 weeks, 1 month, 2 months, 3 months, 4 months, 5 months, 6 months, 1 year). The anti-cancer treatment (e.g., the chemotherapeutic agent), can be administered weekly, e.g., once weekly, for a period of at least 1 week (e.g., at least 2 weeks, 3 weeks, 4 weeks, 5 weeks, 6 weeks, 7 weeks, 1 month, 2 months, 3 months, 4 months, 5 months, 6 months, 1 year) as appropriate. The anti-cancer treatment (e.g., the chemotherapeutic agent), can be administered according to a dosing regimen for gemcitabine.

### Combination Therapy

The anti-cancer treatment (e.g., the chemotherapeutic agent) can be administered in combination with a second anti-cancer treatment (e.g., a second chemotherapeutic agent). The anti-cancer treatment (e.g., the chemotherapeutic agent, e.g., administered at a dose and/or dosing regimen for the chemotherapeutic agent, e.g., known in the art) can be administered in combination with a second anti-cancer treatment (e.g., a second chemotherapeutic agent e.g., administered at a dose and/or dosing regimen for the chemotherapeutic agent, e.g., known in the art)). The second anti-cancer agent can be administered simultaneously or sequentially with the first anti-cancer treatment (e.g., the chemotherapeutic agent). The second anti-cancer treatment can also include a treatment co-formulated with a composition described herein (e.g., a first anti-cancer treatment (e.g., a chemotherapeutic agent).

The second anti-cancer treatment can include, but is not limited to, a chemotherapeutic agent surgery, or radiation therapy. The second anti-cancer treatments can be a chemotherapeutic agent known in the art or described herein. The chemotherapeutic agent can include, nucleoside analogs, cytotoxic agents, MEK inhibitors, a targeted therapy, a tyrosine kinase inhibitor, a proteasome inhibitor, a protease inhibitor, an anti-angiogenic, an anti-fibrotic, an anti-proliferative compound, a steroid, a biologic immunomodulator, a monoclonal antibody, an antibody fragment, an aptamer, a siRNA, an antisense molecule, a fusion protein, a cytokine, a cytokine receptor, a bronchodialator, a statin, an anti-inflammatory agent (e.g. methotrexate), an NSAID hormone treatment, a kinase inhibitor, a tyrosine kinase inhibitor, a vaccines, and/or other immunotherapies.

The anti-cancer treatment (e.g., the chemotherapeutic agent) can be gemcitabine (e.g., administered at a dose and/or dosing regimen for gemcitabine, e.g., known in the art or described herein), and the second anti-cancer agent (e.g., the second chemotherapeutic agent) can be abraxane (e.g., administered at a dose and/or dosing regimen for abraxane, e.g., known in the art described herein).

### Cancers

The compositions described herein can be used to treat a subject. As used herein, a subject is a human.

The compositions provided herein can be used, for example, to treat or prevent a cancer (e.g., a cancer, e.g., a carcinoma or other solid or hematological cancer, a cancer metastases) in a subject. As used herein, the term “cancer” is meant to include all types of cancerous growths or oncogenic processes, metastatic tissues or malignantly transformed cells, tissues, or organs, irrespective of histopathologic type or stage of invasiveness. Methods and compositions disclosed herein are particularly useful for treating, or reducing the size, numbers, or rate of growth of, metastatic lesions associated with cancer.

In some embodiments, the cancer is selected from the group consisting of pancreatic cancer, breast cancer (e.g., triple negative breast cancer, inflammatory breast cancer), esophageal cancer, glioma, stomach cancer, lung cancer, and colorectal cancer. In some embodiments, the cancer is pancreatic cancer. In some embodiments, the cancer is esophageal cancer. In some embodiments, the cancer is breast cancer (e.g., triple negative breast cancer, or inflammatory breast cancer). In some embodiments, the cancer is glioma (e.g., glioblastoma). In some embodiments, the cancer is stomach cancer. In some embodiments, the cancer is lung cancer (e.g., non-small cell lung cancer or small cell lung cancer). In some embodiments, the cancer is colorectal cancer (e.g., colon cancer or rectal cancer).

In some embodiments, the cancer is a metastatic cancer. In some embodiments, the cancer is metastatic pancreatic cancer. In some embodiments, the cancer is metastatic esophageal cancer. In some embodiments, the cancer is metastatic breast cancer. In some embodiments, the cancer is metastatic triple negative breast cancer. In some embodiments, the cancer is metastatic inflammatory breast cancer. In some embodiments, the cancer is metastatic glioma (e.g., metastatic glioblastoma). In some embodiments, the cancer is metastatic stomach cancer. In some embodiments, the cancer is metastatic lung cancer (e.g., metastatic non-small cell lung cancer or metastatic small cell lung cancer). In some embodiments, the cancer is metastatic colorectal cancer (e.g., metastatic colon cancer or metastatic rectal cancer).

Exemplary breast cancers can include triple negative breast cancer, basal-like breast cancer, claudin-low breast cancer, invasive, inflammatory, metaplastic, and advanced Her-2 positive or ER-positive cancers resistant to therapy.

Other cancers include but are not limited to, renal cancer, liver cancer, thyroid cancer, ovarian cancer; prostate cancer, head and neck cancer, a hematological cancer (e.g., a leukemia, e.g., acute lymphoblastic leukemia, acute myelogenous leukemia, T cell leukemia, multiple myeloma; a lymphoma, e.g., diffuse large B cell lymphoma), a skin cancer (e.g., melanoma, squamous cell carcinoma), brain cancer, abdominal cancer, gastrointestinal cancer, liver cancer, neuroblastoma, osteosarcoma, ovarian cancer, retinoblastoma, retinal cancer, bladder cancer, cervical cancer, endometrial cancer, uterine cancer, nasopharyngeal carcinoma, and testicular cancer.

In some embodiments, the cancer is one in which the standard of care for the cancer (in this embodiment, the anti-cancer treatment) is associated with the inducement of one or more of the following properties in at least one cancer cell: (a) a mesenchymal cell phenotype (e.g., as characterized by (e.g., increased vimentin expression and/or decreased E-cadherin expression compared to a reference standard (e.g., expression of the marker in a sample from a cancer which has not been treated with the standard of care))); (b) a cancer stem cell phenotype (e.g., as characterized by (e.g., increased expression of one or more embryonically expressed gene (e.g., increased SOX2, OCT-4, and/or Nanog expression compared to a reference standard), increased CD133, CD44, CD24, THY1, EpCAM, ALDH, and/or ABCB5 expression; compared to a reference standard (e.g., expression of the marker in a sample from a cancer which has not been treated with the standard of care))); or (c) an invasive or metastatic cancer cell phenotype (e.g., as characterized by (e.g., increased AKT signaling, increased Rac signaling, increased Rho signaling, increased ROBO signaling, increased expression of one or more gene associated with metastasis; compared to a reference standard (e.g., expression of the marker in a sample from a cancer which has not been treated with the standard of care))).

### Biomarkers

Methods described herein can include evaluating or acquiring an evaluation of a subject (or a sample from the subject), for one or more of the following properties: (a) modulation of a marker associated with a mesenchymal cell phenotype (e.g., increased vimentin and/or decreased E-cadherin expression compared to a reference standard); (b) modulation of a marker associated with a cancer stem cell phenotype (e.g., increased expression of one or more embryonically expressed gene (e.g., increased SOX2, OCT-4, and/or Nanog expression compared to a reference standard), increased CD133, CD44, CD24, THY1, EpCAM, ALDH, and/or ABCB5 expression compared to a reference standard); or (c) modulation of a marker associated with an invasive or metastatic cancer phenotype (e.g., increased AKT signaling, increased Rac signaling, increased Rho signaling, increased ROBO signaling, increased expression of one or more gene associated with metastasis compared to a reference standard).

The decision can be a decision of whether to administer, continue to administer, discontinue administration, and/or alter the dosage of a treatment, e.g., a treatment described herein, e.g., a LMWH preparation (e.g., M402) and/or an anti-cancer treatment (e.g., a chemotherapeutic agent). For example, if the marker is found to be modulated (e.g., increased or decreased), a treatment, e.g., a LMWH described herein (e.g., M402) and/or an anti-cancer treatment (e.g., a chemotherapeutic agent), can be administered. Thus, measurement of the marker in the subject can determine whether the subject receives a treatment, e.g., a LMWH described herein (e.g., M402) e.g., in combination with the anti-cancer treatment. Measurement of the marker in the subject can determine whether a treatment, e.g., a LMWH described herein (e.g., M402) and/or an anti-cancer treatment (e.g., a chemotherapeutic agent), will be administered, continued to be administered, discontinued, and/or administered at an altered dosage.

Methods of evaluating a marker described herein may be assessed by any of a wide variety of well known methods for detecting expression of a transcribed molecule, gene or protein. Non-limiting examples of such methods include immunological methods for detection of secreted, cell-surface, cytoplasmic, or nuclear proteins, protein purification methods, protein function or activity assays, nucleic acid hybridization methods, nucleic acid reverse transcription methods, and nucleic acid amplification methods.

In certain embodiments, a marker described herein is characterized by a measure of gene transcript (e.g., mRNA), by a measure of the quantity of translated protein, or by a measure of gene product activity. Marker expression can be monitored in a variety of ways, including by detecting mRNA levels, protein levels, or protein activity, any of which can be measured using standard techniques. Detection can involve quantification of the level of gene expression (e.g., genomic DNA, cDNA, mRNA, protein, or enzyme activity), or, alternatively, can be a qualitative assessment of the level of gene expression, in particular in comparison with a control level. The type of level being detected will be clear from the context.

### Monitoring

The methods described herein, e.g., methods of treatment, can further include the step of monitoring the subject, e.g., for a change (e.g., an increase or decrease) in one or more of: tumor size; levels of a cancer marker, for a patient with cancer; the size or rate of appearance of new lesions, e.g., in a scan; the appearance of new disease-related symptoms; the size of soft tissue mass, e.g., a decrease or stabilization; changes in blood flow measured by imaging technology; survival; progression-free survival; quality of life, e.g., amount of disease associated pain, e.g., bone pain; or any other parameter related to clinical outcome. The subject can be monitored in one or more of the following periods: prior to beginning of treatment; during the treatment; or after one or more elements of the treatment have been administered. Monitoring can be used to evaluate the need for further treatment with the same composition or for additional treatment with additional agents. Generally, a decrease in one or more of the parameters described above is indicative of the improved condition of the subject.

### OTHER EMBODIMENTS

This invention is further illustrated by the following examples that should not be construed as limiting. The contents of all references, patents and published patent applications cited throughout this application are incorporated herein by reference.

### EXAMPLES

### Example 1. Accelerated Genetically Engineered Mouse Model for Pancreatic Cancer

Several genetically engineered mouse models (GEMMs) used to study the efficacy and mechanism of action of drug treatments for pancreatic cancer have been reported. The KPC (pdx-1-Cre; Kras^{LSL-G12D/+}; Trp53^{LSL-R172H/+}) model recapitulates the development of human pancreatic adenocarcinoma; however, it poses logistical challenges as mice have a median tumor latency period of about 5 months (Hingorani S R, et al, Cancer Cell. 2005; 7:469-83). The accelerated model using the homozygous Trp53^{FLOX} allele instead of the heterozygous Trp53^{LSL-R172H} allele shows a more differentiated histological phenotype but lacks the strong desmoplastic response, the dense neoplastic stroma characteristic of PDA that contributes to poor delivery of chemotherapeutics and drug resistance (Singh M et al, Nature biotechnology. 2010; 28:585-93).

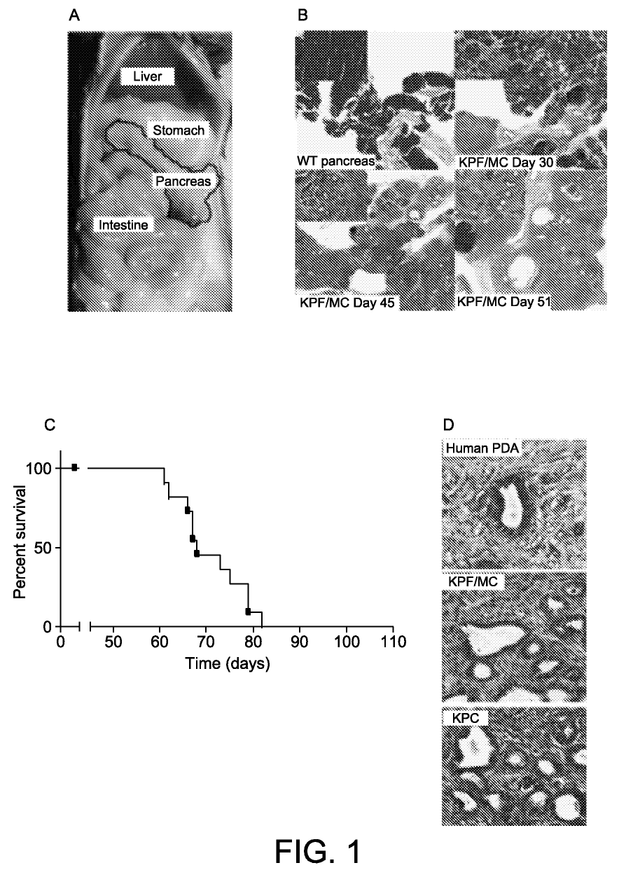

To recapitulate the stromal phenotype of KPC mice in a more accelerated model, the KPFMC (pdx-1-Cre; Kras^{LSL-G12D/+}; Trp53^{LSL-R172H/FLOX}) model was developed as described herein. Homogeneous loss of heterozygocity (LOH) of the Trp53 locus was induced by replacing the wild type allele with the Trp53^{FLOX} allele (Olive K P, et al, Cell. 2004; 119:847-60). LOH of the Trp53 locus is a required event for tumorigenesis in the KPC model as primary pancreatic tumor cell lines from KPC tumors have all lost wild type Trp53 (Hingorani S R, et al, Cancer Cell 2005; 7:469-83.). In the KPFMC mouse model, LOH of the Trp53 locus is no longer a stochastic event and malignant transformation occurs throughout the entire pancreas (FIG. 1A). Tumor development is highly accelerated and 30 days after birth all pancreata show evidence of malignant tumors (FIG. 1B). Due to the rapid tumor growth, the median survival of these mice is approximately 60 days (FIG. 1C). Although the survival of KPFMC mice significantly differs from that of KPC mice, the histology of KPC and KPFMC derived tumors is comparable. Tumors of both models recapitulate the histology of human pancreatic cancer (FIG. 1D). Thus, the KPFMC mouse model represents an accelerated KPC model that can be used for drug intervention studies.

### Example 2. Pretreatment with and Subsequent Administration of M402 in Combination with Gemcitabine Increases Survival Relative to Gemcitabine Monotherapy in Pancreatic Ductal Adenocarcinoma (PDA)

PDA is an almost uniformly lethal disease; and generally considered to be refractory to chemotherapy (Vincent A et al, Lancet. 2011; 378: 607-20). Although adjuvant gemcitabine treatment post surgical removal of a pancreatic tumor has been shown to prolong overall survival; gemcitabine monotherapy in the absence of surgery has failed to show an extensive survival benefit (Oettle H, et al, JAMA 2013; 310:1473-81).

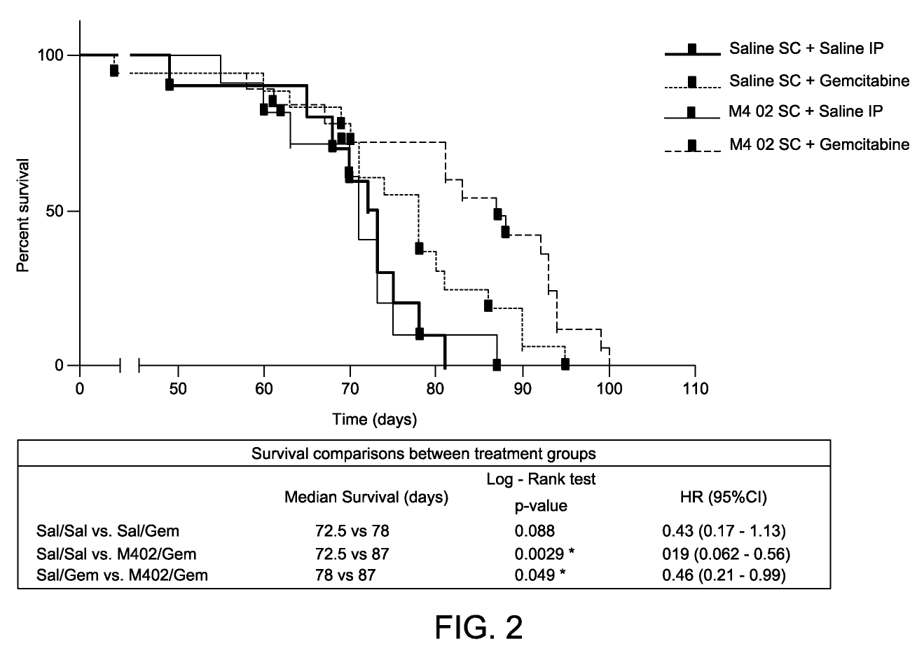

KPFMC mice (pdx-1-Cre; Kras^{LSL-G12D/+}; Trp53^{LSL-R172H/FLOX}) mice were treated with M402 prior to treatment with gemcitabine. M402 was administered subcutaneously, via an osmotic minipump, on day 30 post birth at 40 mg/kg/day for at least four weeks. Gemcitabine was subsequently administered intraperitoneally twice a week at 50 mg/kg starting on day 45 after birth. As shown in FIG. 2, M402 pretreatment followed by gemcitabine provided the highest survival level in KPFMC mice relative to other treatments, including gemcitabine monotherapy. This observation supports that pretreatment with and subsequent administration of M402 in combination with gemcitabine increases survival relative to gemcitabine monotherapy.

### Example 3. Pretreatment with and Subsequent Administration of M402 in Combination with Gemcitabine Reduces Gemcitabine-Monotherapy Induced Invasion in PDA

Gemcitabine Monotherapy Increases Wnt/β-Catenin Signaling and Induces an Invasive Tumor Phenotype.

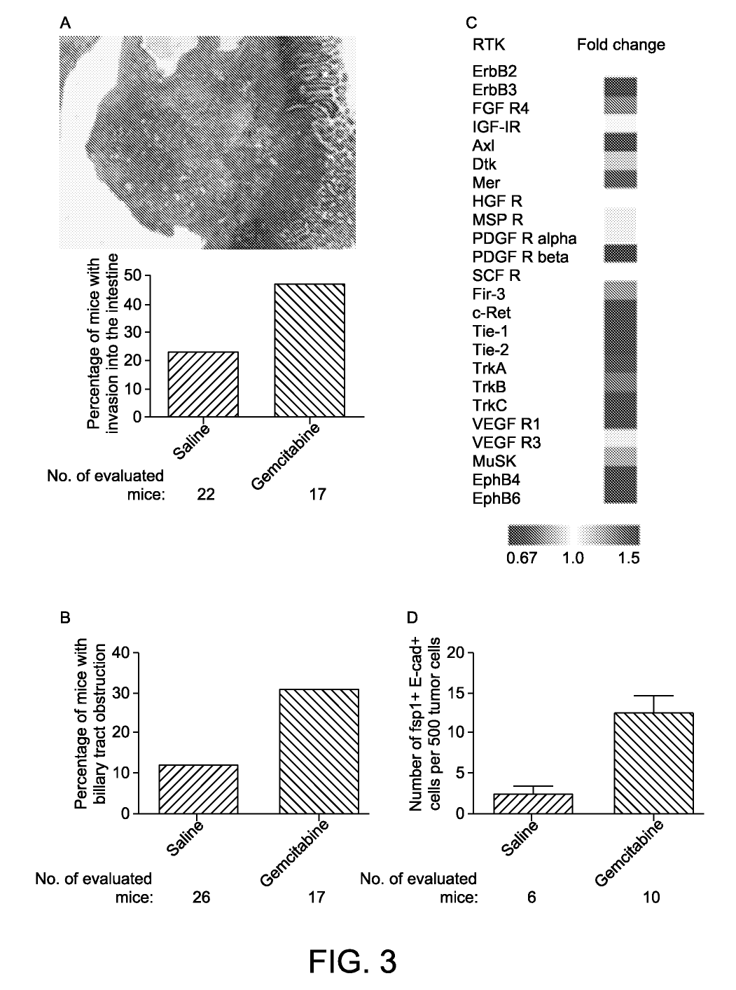

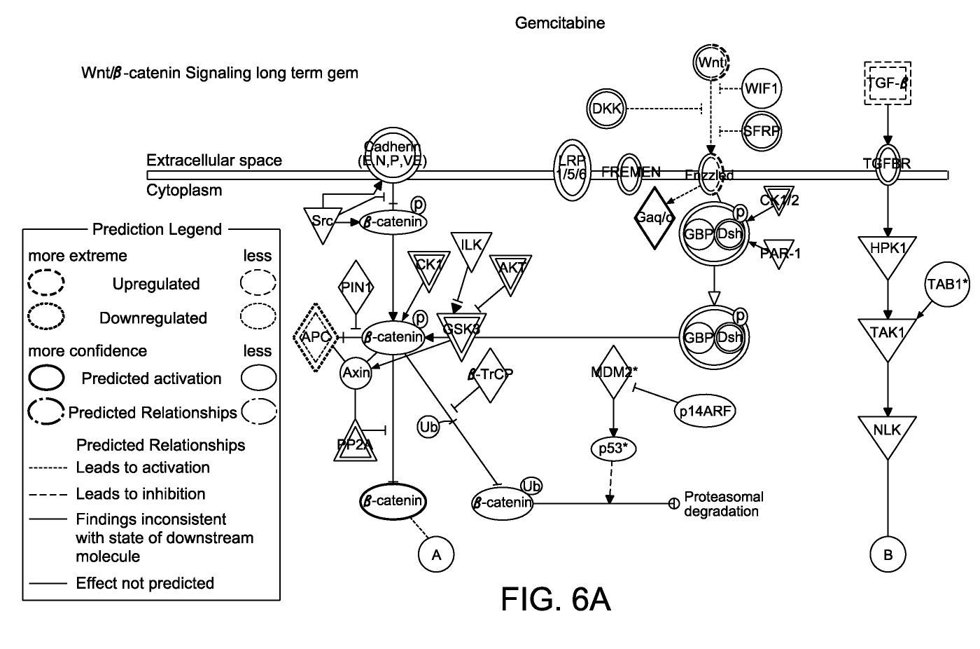

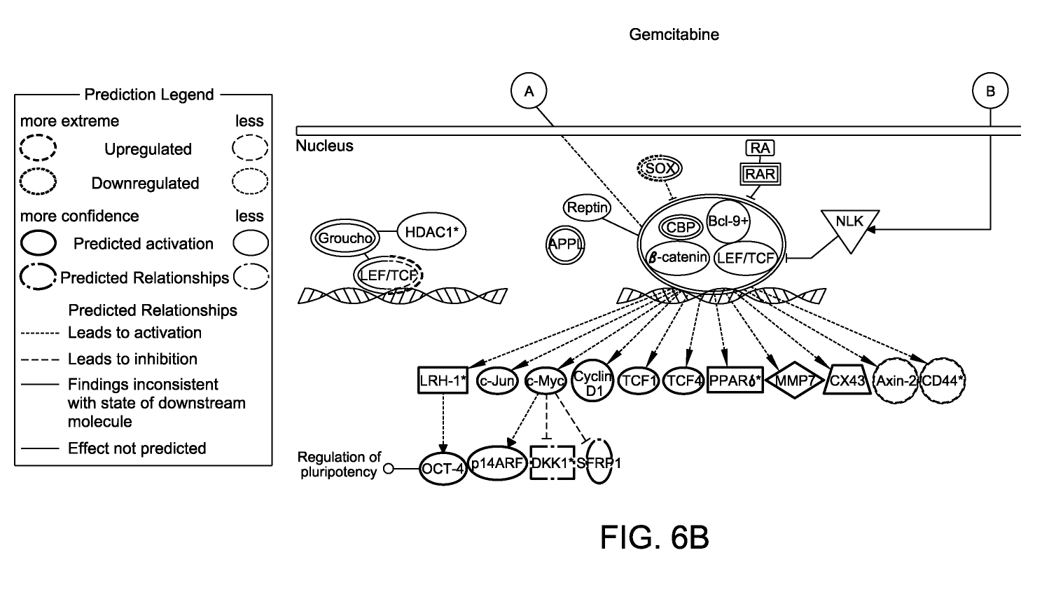

Tumor cells having increased Wnt/β-catenin signaling are known to be invasive. As represented in FIGS. 6A and 6B, gemcitabine monotherapy increases expression of several members of the Wnt/β-catenin signaling pathway including, LRH-1, c-JUN, c-Myc, Cyclin D1, TCF1, TCF4, PPARδ, MMP1, CX43, Jun-2, CD44, Oct-4, p14ARF, Wnt, β-catenin, Frizzled, CBP, Blc9, LEF/TCF. This observation supports that gemcitabine monotherapy results in an invasive tumor phenotype. To confirm that gemcitabine monotherapy results in an invasive tumor phenotype, a cohort of KPFMC mice were exposed to gemcitabine monotherapy (100 mg/kg), starting at day 45 after birth. The tumors were subsequently harvested at the clinical humane endpoint. Histological examination of the tumors from the gemcitabine treated mice showed increased invasion into the adjacent intestine (FIG. 3A) and obstructive jaundice, suggesting invasion into the biliary tree (FIG. 3B). In addition, gene expression analysis showed an increase in tumor cells undergoing EMT (defined as E-cadherin/Fsp1 double positive cells) in mice treated with gemcitabine, indicating gemcitabine treatment induced EMT (FIG. 3C). This observation supports that gemcitabine monotherapy results in an invasive tumor phenotype.

Further, as shown in FIG. 3D gemcitabine treatment also increased the phosphorylation of several RTKs in the pancreatic tumors, which is reported to indicate tumor cell transition towards an invasive phenotype (Knowles L M, et al, Journal of Cancer. 2013; 4:383-90; and Belting M., et al, Trends in Biochemical Sciences, 2003; 28:145-51). This observation further demonstrates active inducement of an invasive tumor phenotype by gemcitabine.

M402 is Associated with a Less Invasive Tumor Phenotype.

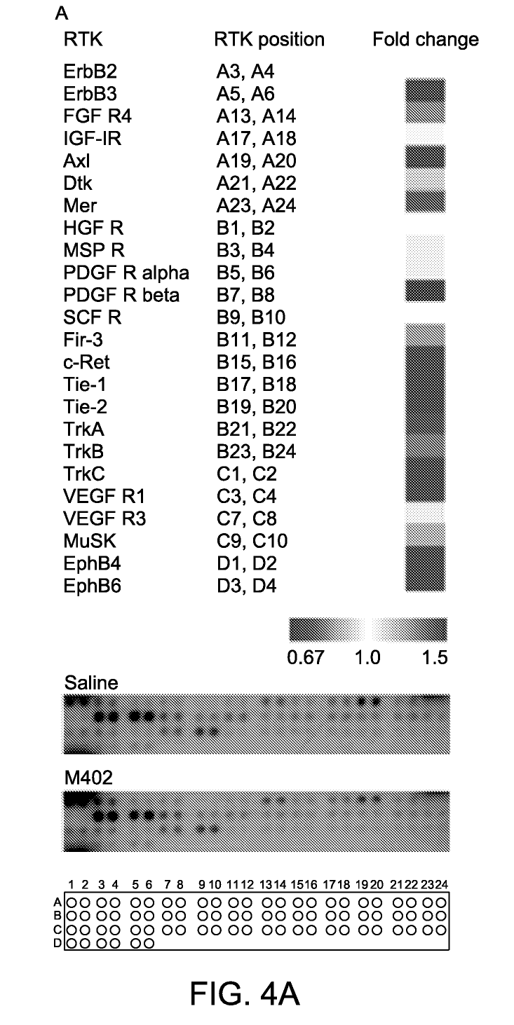

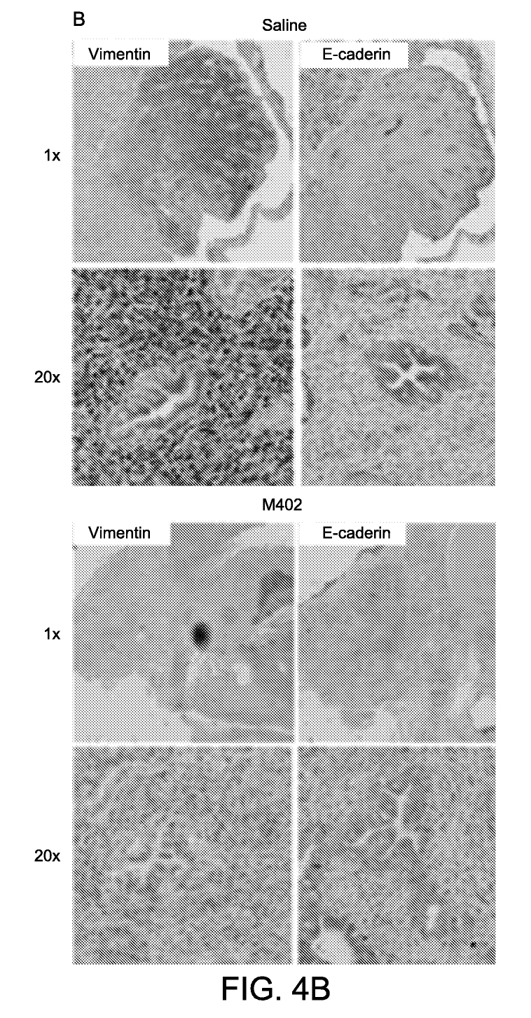

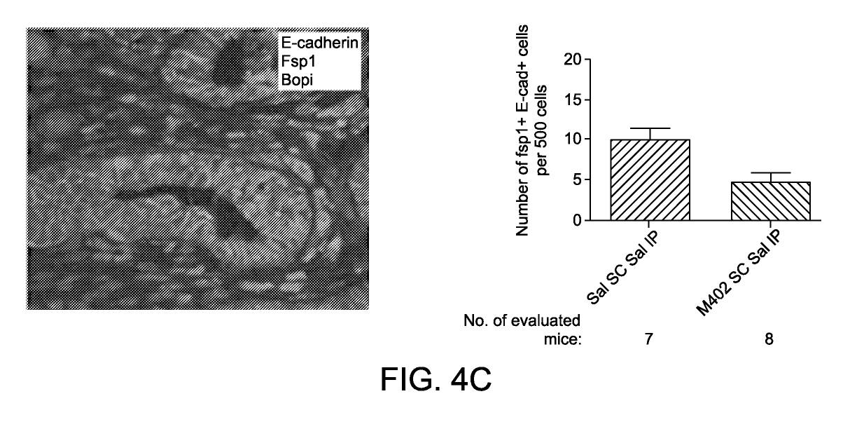

Analysis of tumor samples from M402 treated mice showed a marked alteration in the expression pattern of the EMT markers vimentin and E-cadherin. Saline treated mice demonstrated a large population of vimentin positive cells, whereas the M402 treated mice showed an overall decrease in transition towards an invasive phenotype (FIG. 4B). Immunofluorescence staining of tumor samples derived from mice treated for 30 to 51 days with M402, also showed a decrease in the number of cancer cells undergoing EMT (cells undergoing EMT were defined as E-cadherin/Fsp1 double positive cells) (FIG. 4C). M402 treatment also caused an overall decrease in RTK phosphorylation, e.g., decreased phosphorylation of Ax1 and Tie-1 (FIG. 4A).

Pretreatment with and Subsequent Administration of M402 in Combination with Gemcitabine Reduces Gemcitabine Monotherapy Induced Wnt/β-Catenin Signaling and the Invasive Tumor Phenotype.

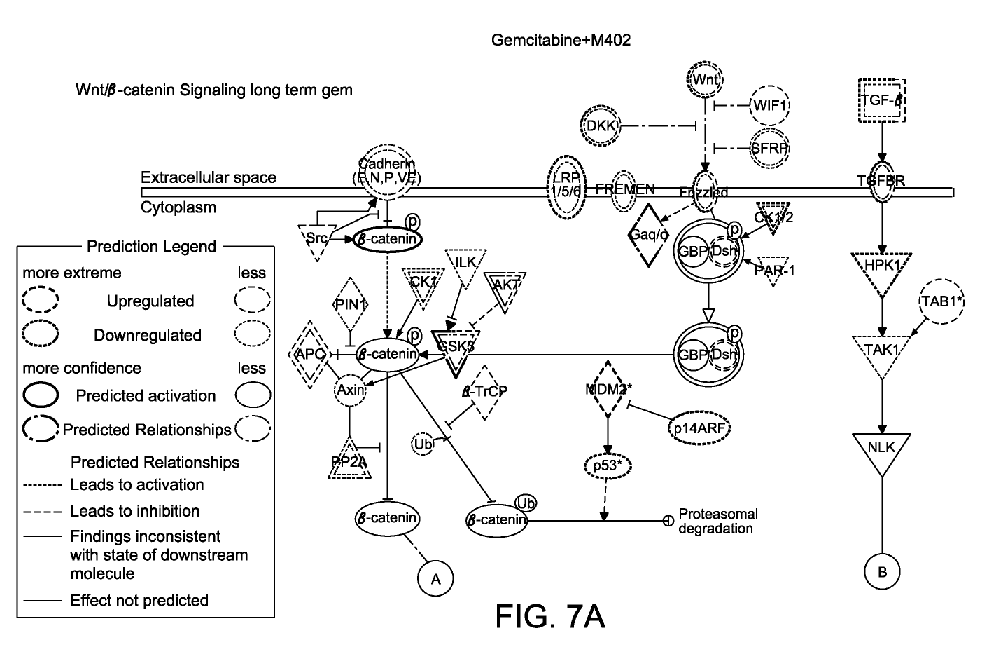

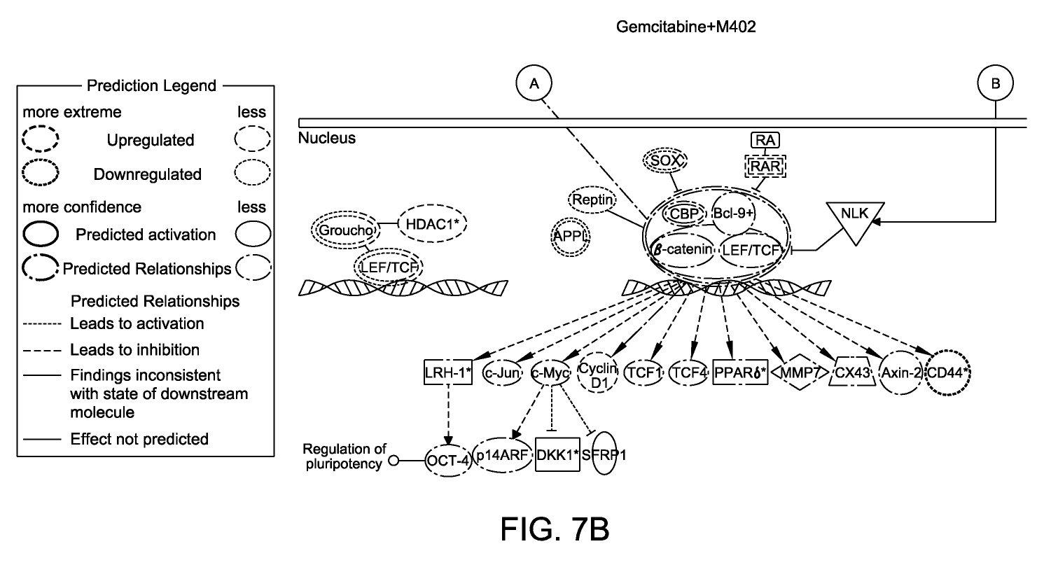

Pretreatment with M402 reduced the gemcitabine induced Wnt/β-catenin signaling, e.g., decreasing expression of several members of the Wnt/β-catenin signaling pathway including, e.g., TGFβ, TGFβR, HPK1, TAK1, CKK, LRP1/5/6, KREMEN, SFRP, CK1/2, PAR1, p53, p14ARF, cadherin, PIN1, CK1, ILK, AKT, GSK3, Axin, PP2A, SOX, REPTIN, APPL, Grouche, LEF/TCF, LRH-1, c-JUN, c-Myc, TCF1, TCF4, PPARδ, MMP1, CX43, Jun-2, CD44, Oct-4, p14ARF, Wnt, β-catenin, Frizzled, CBP, Blc9, LEF/TCF (FIGS. 7A and 7B). This observation supports that M402 inhibits the gemcitabine induced invasive tumor phenotype.

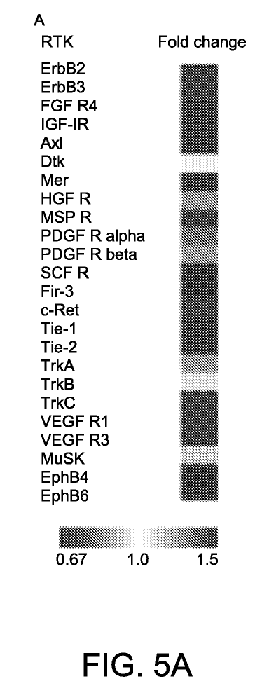

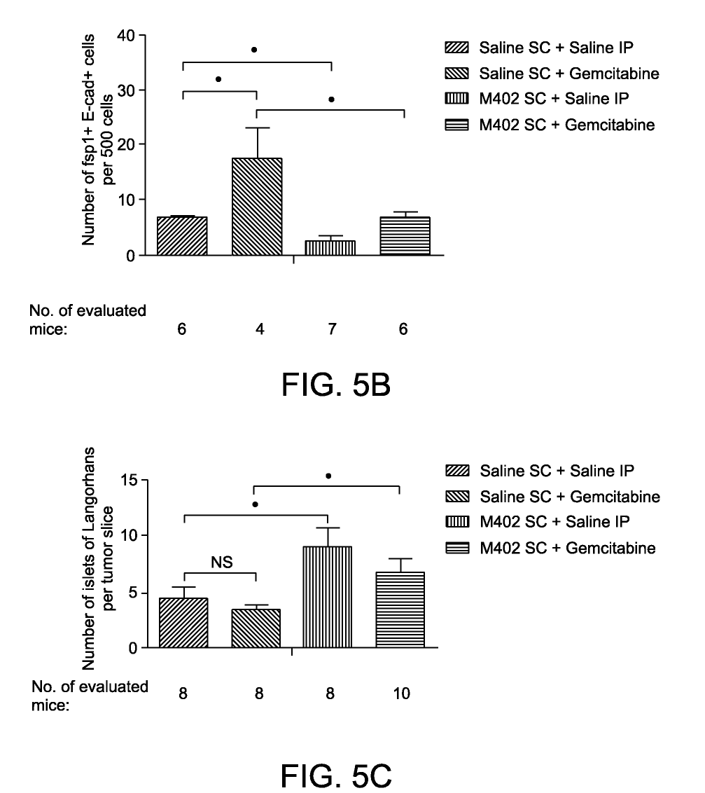

In addition, RTK Ax1 and Tie-2 phosphorylation induced by gemcitabine; was inhibited by M402; and markedly inhibited in the combination gemcitabine/M402 treatment (FIG. 5A), suggesting M402 mediated amelioration in the gemcitabine induced invasive phenotype. In addition, tumors derived from mice treated with M402 and gemcitabine combination therapy showed a decrease in the number of tumor cells undergoing EMT compared to gemcitabine monotherapy (cells undergoing EMT were defined as E-cadherin/Fsp1 double positive cells), suggesting M402 is able to mitigate the development of the invasive/chemotherapy resistant tumor phenotype induced by gemcitabine (FIG. 5B).

As tumor development progresses, tumor cell invasion results in local destruction of normal pancreatic endocrine tissues, including islet of Langerhans cells. In the KPFMC model, Islets of Langerhans cells are randomly distributed throughout the pancreas and do not undergo malignant transformation. To assess the level of destruction of the normal pancreatic tissue, the number of insulin positive islets of Langerhans cells was determined in samples from mice treated from 30 to 51 days with M402, gemcitabine, or M402 pretreatment followed by combination treatment of gemcitabine and M402. The number of intact islet cells was significantly higher in the groups treated with M402 alone and M402 pretreatment followed by combined gemcitabine treatment, when compared to gemcitabine alone and saline treated tumors (FIG. 5C), suggesting M402 counteracts the normal cell destruction caused by gemcitabine.

### Example 4. Pretreatment with and Subsequent Administration of M402 in Combination with Gemcitabine and Abraxane Reduces Tumor Weight and Chemotherapy Induced Reduction in Immune Cells

Pretreatment with and Subsequent Administration of M402 in Combination with Gemcitabine Reduces Tumor Weight.

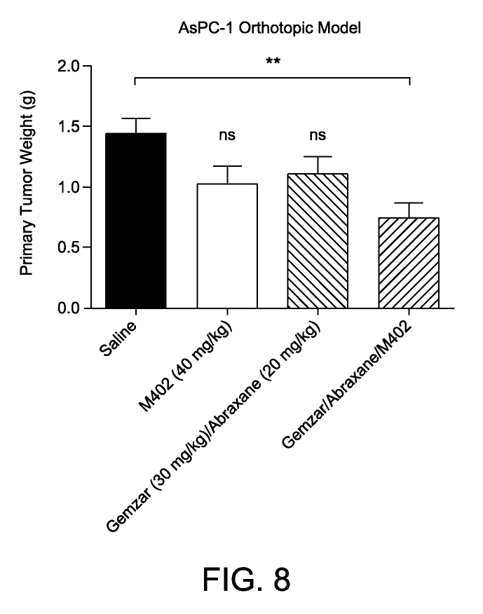

AsPc-1 human pancreatic adenocarcinoma cells were injected into the pancreata of immunodeficient (Nu/Nu CD-1) mice at eight weeks of age. The mice were pretreated with M402 prior to administration of gemcitabine or gemcitabine and abraxane. Specifically, M402 was administered starting on day 18 post cell transfer at 40 mg/kg via osmotic pump; gemcitabine was administered intraperitoneally twice a week at 30 mg/kg starting on day 22; and abraxane was administered intravenously twice a week at 20 mg/kg starting on day 22. Combination treatments were administered on day 22, 25, 29, and 32 for a total of four doses. Pretreatment with M402 followed by combination treatment of M402, gemcitabine (Gemzar), and abraxane significantly reduced primary tumor weight compared to controls (FIG. 8).

Pretreatment with and Subsequent Administration of M402 in Combination with Gemcitabine Rescues Chemotherapy Induced Reduction in Immune Cells.

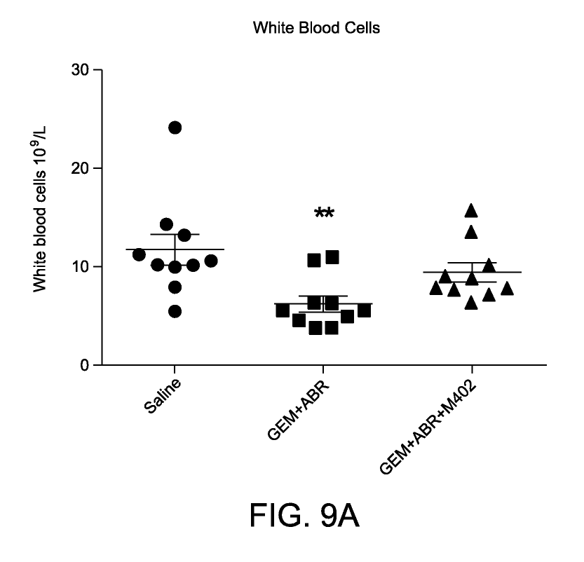

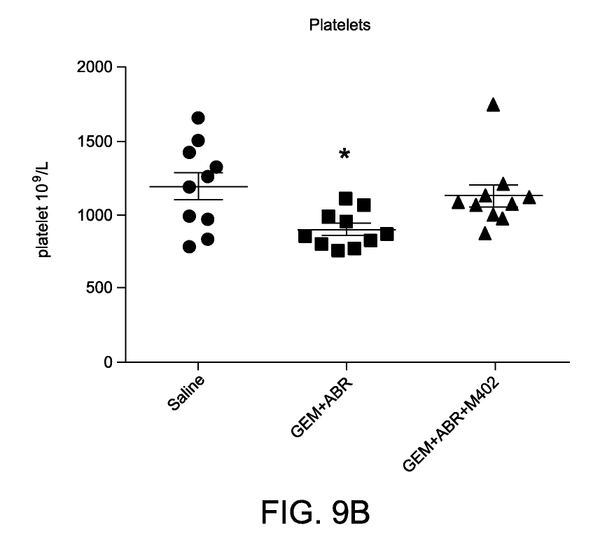

Capan-2 human pancreatic adenocarcinoma cells were injected into the pancreta of immunodeficient (Nu/Nu CD-1) mice at eight weeks of age. The mice were pretreated with M402 prior to administration of gemcitabine or gemcitabine and abraxane. Specifically, M402 was administered starting on day 28 post cell transfer at 40 mg/kg via osmotic pump; gemcitabine was administered intraperitoneally twice a week at 30 mg/kg starting on day 42; and abraxane was administered intravenously twice a week at 20 mg/kg starting on day 42. Combination treatments were administered on day 42, 45, 49, 52, and 55 for a total of five doses. VetScan analysis showed gemcitabine and abraxane decreased white blood cells (WBCs) and platelet (PLT) counts, while pretreatment with M402 followed by combination treatment of M402, gemcitabine (Gemzar), and abraxane rescued the levels of WBCs and PLTs (FIG. 9A and FIG. 9B).

1. A method of treating a cancer in a subject, the method comprising administering at least one dose of a LMWH preparation (e.g., a M402 preparation) described herein, and administering an initial dose of an anti-cancer treatment (e.g., a chemotherapeutic agent) that increases wnt/β-catenin signaling, wherein the LMWH preparation (e.g., the M402 preparation) is administered prior to the administration of the initial dose of the anti-cancer treatment (e.g., the chemotherapeutic agent).

2-120. (canceled)
